(19) United States
(12) Patent Application Publication
(10) Pub. No.: US 2021/0030283 A1
(43) Pub. Date: Feb. 4, 2021

(54) SYSTEMS AND METHODS FOR PROVIDING REAL-TIME PERFUSION GUIDED TARGETS FOR PERIPHERAL INTERVENTIONS

(71) Applicant: Pedra Technology Pte. Ltd., Singapore (SG)

(72) Inventors: Kareen Looi, Singapore (SG); Paul Hayes, Cambridge (GB)

(21) Appl. No.: 16/815,935

(22) Filed: Mar. 11, 2020

Related U.S. Application Data
(60) Provisional application No. 62/816,805, filed on Mar. 11, 2019.

(51) Int. Cl. A61B 5/026; A61B 5/00; G16H 50/30; G16H 50/20

(52) U.S. Cl. CPC A61B 5/0261; G16H 50/20; G16H 50/30; A61B 5/742

(57) ABSTRACT

Disclosed herein are computer-implemented real-time systems and methods for determining success of a revascularization procedure and/or wound healing of a patient, that can involve measuring blood perfusion characteristics utilizing diffuse speckle contrast analysis (DSCA), and determining blood perfusion and vascular health indices predictive of a likely positive or negative patient outcome, and communicating that outcome to an operator utilizing a display, etc.

### PRIORITY CLAIM

This application claims the benefit under 35 U.S.C. § 119(e) as a nonprovisional application of U.S. Prov. App. No. 62/816,805 filed on Mar. 11, 2019, which is hereby incorporated by reference in its entirety.

### BACKGROUND

### Field

This disclosure relates to the measurement of blood flow in tissue, in particular measurement of blood flow in the foot or other extremities.

### Description of the Related Art

The rapidly aging population in the developed world has led to an increasing prevalence of aging-associated degenerative diseases such as peripheral arterial disease and Type 2 diabetes. The manifestations of these include tissue ischemia, chronic wounds and diabetic foot ulcers, where lack of appropriate treatment may lead to infection, gangrene and, in the case of foot ischemia, partial or complete amputation of one or both feet.

Peripheral arterial di cease (PAD) is a progressive disease in which narrowed or obstructed arteries reduce blood flow to the limbs. PAD can result from atherosclerosis, inflammatory processes leading to stenosis, an embolism, or thrombus formation, and is associated with smoking, diabetes, dyslipidemia, and hypertension. PAD can if untreated result in critical limb ischemia (CLI), in which blood flow to the limb (usually the legs and feet) is compromised to such an extent that tissue damage ensues with consequent ulceration, gangrene or loss of the limb. Patients with PAD are also at a disproportionately high risk of other cardiovascular diseases like myocardial infarction and stroke and of death as a result of these conditions. With the incidence of diabetes increasing worldwide, treatment of CLI and prevention of disability and of limb loss from it has become a significant health priority.

Peripheral vascular intervention procedures using endovascular (minimally invasive) intervention, open surgery or a combination of the two are currently the only methods available to restore perfusion to the limbs in patients with PAD. Medical management can help only to delay the progression of the disease, if at all. However, clinicians currently lack the intraoperative tools to properly assess perfusion in the affected tissue, usually in the feet, in real-time to reliably guide the conduct of the interventional procedure. Existing technologies that measure blood perfusion include skin perfusion pressure (SPP), duplex ultrasound (DUX), and transcutaneous oxygen monitoring (TCOM). Each of these techniques suffers from one or more disadvantages. SPP only provides perfusion data at the skin dermis level, requires the skin temperature to be normalized to 44° C., is affected by skin pigmentation and is unreliable with patients with edema. SPP also requires the use of a pressure cuff, which further limits its utility as a real-time perfusion assessment tool during peripheral vascular interventions. DUX does not assess tissue perfusion but instead measures blood flow in large vessels (>1.5 mm). TCOM requires the patient to be placed on hyperbaric oxygen, making it incompatible with the cath lab/operating room. Furthermore, TCOM does not provide real time revascularization data as it takes about 4 to 6 weeks for the measurements to equilibrate.

Accordingly, there is a need for noninvasive, real-time measurement of blood perfusion in a range of blood vessel sizes and in the tissue supplied by these vessels. In particular, there is a need for noninvasive, real-time measurement of blood perfusion in the foot that can be reliably performed as the interventional procedure proceeds and be used to inform the decision making during the procedure.

Ischemia is a condition where a restriction of blood supply to tissues leads to a shortage in oxygen and glucose, resulting in irreversible damage to tissues. If discovered too late, reperfusion of blood by various treatment options, thrombolytic or surgical, will only further increase the damage to the tissue as opposed to rescuing the tissue. For example, one of the most common sites of ischemia is the foot. In this case, early detection and diagnosis of an ischemic foot at risk is imperative, before the damage becomes irreversible. Currently, the most common way to diagnose an ischemic foot is ABI (Ankle Brachial Index) which compares the blood pressure in the arm with that at the ankle. An ABI measurement less than 0.9, in some cases, is indicative of an ischemic foot. However, ABI measurements are highly dependent on operator protocol, i.e. different values can be obtained when measurements are obtained with the subject in a seated or supine position, or when the operator uses a different measurement protocol/equipment. ABI also produces falsely elevated measurements in calcified vessels of patients who have diabetes mellitus, are receiving hemodialysis, or if there is an extensive distal arterial lesion below the ankle (Yamada et al, J Vasc Surg 2008; 47: 318-23).

A chronic wound is a non-healing wound that shows little or no improvement after four weeks or does not heal in eight weeks. In practice, patients may present with chronic wounds that remain open for over a year. Around the world, there are 37 million people who suffer from chronic wounds, mostly on the lower limbs. In the US alone, chronic wounds have affected 6.5 million patients and accounted for $1.4 billion in spending in 2010. Since chronic wounds are associated with the diseases of aging, such as diabetes and obesity, the healthcare need for chronic wound management is rising together with the rise in aged populations in the developed world. The early diagnosis of a chronic ischemic wound on lower limbs is particularly important, as it has a major impact in determining whether conservative wound management (e.g., bandages and moist dressings) would be sufficient, or whether more aggressive therapies are required to forestall further wound deterioration that may culminate in amputation.

Conservative therapy for wounds (e.g., bandages, moist dressings) can suffice to facilitate wound healing if the blood perfusion around the wound tissue is not compromised beyond the minimal threshold for passive healing to occur. In cases where the perfusion is compromised, however, the inappropriate use of conservative wound therapy causes a time lag between the first presentment of a wound in a clinical setting to an effective therapy commensurate with the seriousness of the wound condition.

The single most important determinant of tissue viability in a wound is its blood supply. The ability to assess the blood perfusion around the wound bed allows clinical decisions to be made regarding either (a) continuation of conservative therapy if tissue is viable or, (b) if blood perfusion is too severely compromised for successful conservative therapy, to progress early to more advanced wound care products like chemical debriding agents, or advanced wound therapies such as topical negative pressure, hyperbaric oxygen therapy (“HBOT”), etc. In appropriate cases, the patient can be directed to revascularization by peripheral interventional procedures. Hence, a blood perfusion monitor that can facilitate the early streaming of patients into conservative versus aggressive wound therapies is highly desirable.

HBOT involves the administering of oxygen at levels 2-2.5 times sea level in a hyperbaric chamber. A patient may be prescribed up to 40 sessions of HBOT, with typically 3-4 sessions per week, in order to maximize the delivery of oxygen to chronic wound tissue. Such therapy is expensive and is not without risk; its side effects include ear and sinus barotrauma, paranasal sinuses and oxygen toxicity of the central nervous system. (Aviat Space Environ Med. 2000;71(2):119-24.) Moreover, a retrospective study of 1144 patients (Wound Rep Reg 2002; 10:198-207) indicated that 24.4% of chronic wound patients received no benefit from it. Therefore, a diagnostic device to better predict the success of HBOT in chronic wound treatment will help to avoid unnecessary and unhelpful therapy, and obtain significant cost savings in the healthcare system.

In foot ischemia cases where amputation is required, there is a need for a new diagnostic tool that can better guide decisions regarding the amputation level, by predicting the potential success of amputation wound healing. Amputation is typically performed on patients with severe limb ischemia who cannot be treated with reconstructive vascular surgery, patients with diabetic foot ulcers or venous ulcerations. Approximately 85-90% of lower limb amputations in the developed world are caused by peripheral vascular disease and poor wound healing accounts for 70% of the complication cases that arise from amputation. Due to the lack of optimal tools to predict amputation healing, physicians have to make subjective judgments on the best site for amputation, and since the bias is to maximize limb preservation, it is not uncommon for a patient to require a subsequent amputation higher up the leg when the first amputation wound is unable to heal. The healing rate of below-knee amputation ranges between 30 and 92%, with a re-amputation rate of up to 30%. Thus, an accurate tool for predicting successful amputation healing is needed to help doctors more accurately determine the site of amputation that will result in maximal limb preservation while avoiding the trauma and cost of a revision amputation.

Generally in surgical procedures, particularly in plastic and reconstructive surgery, tissue flaps are used to cover wound defects. These may be either pedicled flaps (i.e. have a vascular pedicle of their own that supplies blood to the flap) or free-flaps that need microvascular connections with the recipient site to ensure adequate blood supply. Both types of flaps are crucially dependent on the blood perfusion within them for the flaps to survive. Flap perfusion needs close monitoring especially in the first few hours to days after the reconstruction procedure and early detection of loss of perfusion will help to direct the patient for further surgical procedures as needed to ensure continued flap viability. It will thus be useful if a diagnostic tool can potentially be used to monitor flap blood perfusion continuously in the post-operative period and prevent flap loss due to delayed detection of flap ischemia.

Currently, diagnostic devices on the market for wound care include duplex ultrasound (for example, as described in EP0814700 A1), transcutaneous oxygen monitoring (TCOM or TcPO₂) (for example, as described in WO1980002795 A1), and skin perfusion pressure (SPP) (for example, as described in CA2238512 C), each of which suffer severe disadvantages that limits their effectiveness in administering the right therapy to chronic wound patients. Duplex ultrasound only measures blood flow in large vessels (>1.5 mm). TCOM measurements are not optimally correlated with the status of the wound. (Wounds 2009;21(11):310-316). This is especially so as TCOM measurements are influenced by many factors including local edema, anatomical localization, thickness of the epidermal stratum corneum, and leg dependency (Figoni et al, J. Rehab Research Development 2006; 43 (7) 891-904). In addition, test results are heavily affected by moisture and temperature levels (Podiatry Today 2012; 25(7) 84-92). Lo et al. (Wounds 2009:21(11) 310-316) report that skin perfusion pressure (measured by laser Doppler) appears to be a more accurate predictor of wound healing versus TcPO₂; however SPP is only able to provide data at limited depth and requires skin temperature to be normalized to 44° C., is sensitive to skin pigmentation and unreliable with edema.

Most recently, the use of diffuse speckle contrast analysis (DSCA) has been developed to measure real-time blood perfusion in tissue depths of up to two centimeters (2 cm), in absolute BFI (“blood flow index”) units, which can also be referred to herein as BPI (“blood perfusion index”). The present disclosure centers in some embodiments on the use of DSCA via a hardware or software processor configured to generate indices predictive of, and that can guide clinical decisions in treating ischemia and other conditions. Systems and methods as disclosed herein can be used or modified for use with U.S. Pat. No. 9,636,025 to Lee et al., and U.S. Pub No. 2015/0073271 A1 to Lee et al., both of which are incorporated by reference in their entireties.

### SUMMARY

In some embodiments, disclosed herein is a computer-implemented real-time method for determining success of a revascularization procedure and/or wound healing of a patient, the method comprising any number of: measuring blood perfusion characteristics utilizing diffuse speckle contrast analysis (DSCA); determining a blood perfusion index (BPI) at a first point in time, determining the blood perfusion index (BPI) at a second point in time, determining a blood perfusion index (BPI) ratio from the BPI by dividing the BPI at the second point in time by the BPI at the first point in time; analyzing the BPI ratio with respect to additional patient characteristics; and/or outputting to a display the BPI ratio and indicia of a likely positive or negative patient outcome.

In some configurations, the additional patient characteristics comprises an absolute value of the BPI.

In some configurations, the method also comprises outputting to a display indicia of a likely positive patient outcome if the BPI ratio is greater than about 1.

In some configurations, the method also comprises outputting to a display indicia of a likely positive patient outcome if the BPI ratio is greater than about 2.

In some configurations, the method also comprises outputting to a display indicia of a likely negative patient outcome if the BPI ratio is less than about 1.

In some configurations, the first point in time is within 5 minutes of a first angioplasty attempt within the revascularization procedure, and after guidewire placement.

In some configurations, the second point in time is within 5 minutes of completion of the revascularization procedure.

In some configurations, the method also comprises outputting to a display indicia of a likely positive patient outcome if the BPI ratio is greater than about 1.1.

In some configurations, the method also comprises outputting to a display indicia of a likely positive patient outcome if the BPI ratio is greater than about 1.2.

In some configurations, the method also comprises outputting to a display indicia of a likely positive patient outcome if the BPI ratio is less than about 0.9.

In some configurations, the method also comprises outputting to a display indicia of a likely positive patient outcome if the BPI ratio is less than about 0.8.

In some configurations, measuring occurs on the skin surface of the patient.

In some configurations, measuring and determining is performed at a location of interest on the patient, and the additional patient characteristics are determined by: identifying a reference location on the patient at a location perfused by different vasculature than the location of interest on the patient; measuring blood perfusion characteristics utilizing diffuse speckle contrast analysis (DSCA) at the reference location; determining a blood perfusion index (BPI) at the first point in time at the reference location, determining the blood perfusion index (BPI) at the second point in time at the reference location, determining a reference blood perfusion index (BPI) ratio from the BPI by dividing the BPI at the second point in time by the BPI at the first point in time, wherein analyzing the BPI ratio further comprises adjusting the BPI ratio at the location of interest based at least in part by the reference BPI.

In some configurations, the reference location on the patient is on an arm, forearm, or torso of the patient.

Also disclosed herein in some embodiments is a computer-implemented real-time system for determining success of a revascularization procedure and/or wound healing of a patient. The system can include any number of: a laser light source; a detector; and a processor configured to electronically perform one or more of the following: receiving measured blood perfusion characteristics from a location of interest utilizing diffuse speckle contrast analysis (DSCA) from the detector; determining a blood perfusion index (BPI) at a first point in time, determining the blood perfusion index (BPI) at a second point in time, determining a blood perfusion index (BPI) ratio from the BPI by dividing the BPI at the second point in time by the BPI at the first point in time; analyzing the BPI ratio with respect to additional patient characteristics; and outputting to a display and electronically indicating the BPI ratio and indicia of a likely positive or negative patient outcome.

In some configurations, the additional patient characteristics comprise an absolute value of the BPI.

In some configurations, the processor is configured to output to a display indicia of a likely positive patient outcome if the BPI ratio is greater than about 1.

In some configurations, the processor is configured to output to a display indicia of a likely positive patient outcome if the BPI ratio is greater than about 2.

In some configurations, the processor is configured to output to a display indicia of a likely negative patient outcome if the BPI ratio is less than about 1.

In some configurations, the processor is configured to output to a display indicia of a likely positive patient outcome if the BPI ratio is greater than about 1.1.

In some configurations, the processor is configured to output to a display indicia of a likely positive patient outcome if the BPI ratio is greater than about 1.2.

In some configurations, the processor is configured to output to a display indicia of a likely positive patient outcome if the BPI ratio is less than about 0.9.

In some configurations, the processor is configured to output to a display indicia of a likely positive patient outcome if the BPI ratio is less than about 0.8.

In some configurations, the processor is further configured to receive blood. perfusion characteristics from a reference location on the patient; determine reference BPI ratios at the first and second points in time at the reference location, and adjust the BPI ratio at the location of interest based at least in part by the reference BPI,

In some embodiments, disclosed herein is a computer-implemented real-time method for determining necessity of a revascularization procedure, the method comprising any number of: measuring blood perfusion characteristics utilizing diffuse speckle contrast analysis (DSCA); determining a blood perfusion index (BPI) over a set period of time; deriving a vascular health index (VHI) from the BPI data; analyzing the VHI with respect to additional patient characteristics; and outputting to a display the VHI and indicia of a likely need for the revascularization procedure.

In some configurations, the additional patient characteristics comprises an absolute value of the BPI.

In some configurations, the method also comprises outputting to a display indicia of a likely need for the revascularization procedure if the VHI is less than about 20.

In some configurations, the method also comprises outputting to a display indicia of a likely need for the revascularization procedure if the VHI is less than about 15.

In some configurations, the method is determined in the outpatient setting.

In some embodiments, also disclosed herein is a computer-implemented. real-time system for determining necessity of a revascularization procedure, the system comprising any number of: a laser light source; a detector; and a processor configured to perform one or more of the following: measuring blood perfusion characteristics utilizing diffuse speckle contrast analysis (DSCA); determining a blood perfusion index (BPI) over a set period of time; deriving a vascular health index (VHI) from the BPI data; analyzing the VHI with respect to additional patient characteristics; and outputting to a display the VHI and indicia of a likely need for the revascularization procedure.

In some embodiments, a system can include, exclude, consist essentially of, or consist of any number of features as set forth in the disclosure.

In some embodiments, a method can include, exclude, consist essentially of, or consist of any number of features as set forth in the disclosure.

### BRIEF DESCRIPTION OF THE DRAWINGS

FIG. 1 illustrates a portable system comprising DSCA technology.

A diagrammatic representation of the DSCA process is shown in FIG. 1AA.

FIG. 1A illustrates the pedal angiosomes.

FIG. 1B illustrates five measurement points on the foot, each corresponding to one of the angiosomes shown in FIG. 1A.

FIG. 1C illustrates the branching of the arteries supplying the pedal angiosomes.

FIGS. 1D-1H illustrate measurement using diffuse optical flow (DOF) sensors at each of the five measurement positions of FIG. 1B.

FIG. 2 is a block diagram of a system for measuring flow of turbid media.

FIG. 3 is a schematic illustration of diffuse light penetration and detection in multi-layer tissue.

FIG. 4 is a graph of autocorrelation functions for different flow rates.

FIG. 5A is a graph of two blood flow indices (BFIs, also known as BPIs) during cuff occlusion protocol.

FIG. 5B is a graph of autocorrelation functions illustrating the derivation of the two BFIs (BPIs) of FIG. 5A.

FIG. 6 is a graph of two BFIs (BPIs) during cuff occlusion protocol.

FIG. 7 illustrates various elements of a perfusion monitoring system, according to some embodiments.

FIG. 7A illustrates an embodiment of a DSCA perfusion monitor console and instrumentation box.

FIG. 7B illustrates embodiments of low-profile sensors.

FIG. 8A shows the raw BFI (BPI) data (raw time series BFI data) measured at the medial plantar section of the foot of two individuals, one healthy versus one with indications of limb ischemic, while FIG. 8B shows the equivalent power spectrum data of the same individuals (Fourier transform of raw time series BFI data.

FIGS. 9A-10 show embodiments of support structures.

FIG. 11 illustrates a plurality of DOF sensors 1000 attached to a patient's foot.

FIG. 12 shows the Flow Transform Level (FTL) relating to the time series BFI, e.g., derivation of FTL from time series DSCA blood flow index (BFI) data, where intensity is measured at a frame rate of 60 Hz.

The standard deviation of 5 minutes of Medial Plantar BFI data sampled at 1 Hz and 2 Hz was calculated. and the resulting ROC curves are shown in FIGS. 13A and 13B. FIG. 13A illustrates the ROC of Standard Deviation of BFI @ 1 Hz; FIG. 13B illustrates the ROC of Standard Deviation of BI @ 2 Hz.

The Standard Deviation of BFI from calcaneal and arm also shows significant difference between healthy and ischemic patients, but not strongly as with the medial plantar. The p-values of three positions are compared in FIGS. 14A-14C, which are box plots of FTLs in the medial plantar, calcaneal, and arm regions, respectively. FIG. 14D illustrates FTL values for a number of patients including healthy and ischemic patient populations.

A study of healthy patients vs. patients with clinically diagnosed PAD or CLI, generated the following AUC graphs, illustrated in FIGS. 14E and 14F.

In one study, the baseline VHI for 20 patients was analyzed as shown in FIG. 14G and as follows.

For the same patients, the median of the average BPI value over the same 5-minute period (taking a visual estimate of the average BPI off the 5-minute chart) is shown in FIG. 14H.

### DETAILED DESCRIPTION

### Diffuse Optical Flow Sensors

A number of techniques exist for characterizing blood flow (which may also be referred to herein as blood perfusion), relying on measuring of diffusion of light. Such techniques include Diffuse Correlation Spectroscopy (DCS) and Diffuse Speckle Contrast Analysis (DSCA). Both DCS and DSCA can be used to measure relative and/or absolute blood flow. Other techniques rely on measuring diffusion of light to detect other characteristics of tissue, such as biochemical composition, concentrations of oxyhemoglobin and deoxyhemoglobin, etc. Such techniques include Diffuse Optical Spectroscopy (DOS), Diffuse Optical Tomography (DOT), and Near-Infrared Spectroscopy (NIRS).

As used herein, “diffuse optical sensor” includes any sensor configured to characterize properties of blood in tissue via measurement of diffuse light. As such, diffuse optical sensors include DCS, DSCA, DOS, DOT, and NIRS sensors. As used herein, the term “diffuse optical flow sensor” includes any sensor configured to characterize blood flow in tissue. As such, diffuse optical flow (DOF) sensors include both DCS and DSCA sensors.

Near-infrared diffuse correlation spectroscopy (DCS) is an emerging technique for continuous noninvasive measurement of blood flow in biological tissues. In the last decade or so, DCS technology has been developed to noninvasively sense the blood flow information in deep tissue vasculature such as brain, muscle, and breast. In contrast to some other blood flow measurement techniques, such as positron emission tomography (PET), single photon emission computed tomography (SPECT), and xenon-enhanced computed tomography (XeCT), DCS uses non-ionizing radiation and requires no contrast agents. It does not interfere with commonly used medical devices such as pacemakers and metal implants. It therefore has potential in cancer therapy monitoring and bedside monitoring in clinical settings.

A DCS system can include a light source such as a laser with a long coherence length, a detector such as a photon-counting avalanche photodiode (APD) or photomultiplier tube (PMT), and an autocorrelator. In various embodiments, the autocorrelator may take the form of hardware or software. As one of the central components of the DCS system, the autocorrelator computes the autocorrelation function of the temporal fluctuation of the light intensity obtained from the detector.

However, DCS can suffer from a long integration time, high cost, and low channel number of simultaneous measurements. One factor contributing to these limitations is dependence on very sensitive photodetector(s) and subsequent autocorrelation calculation. Diffuse Speckle Contrast Analysis (DSCA) is a newer technology that provides an improved flowmetry system enabling cost-effective, real-time measurements using statistical analysis without having to rely on autocorrelation analysis on fast time-series data. This statistical analysis can be implemented either in spatial domain using a multi-pixel image sensor, or in the time domain using slow counter. A multi-pixel image sensor can also be used for time domain analysis such that single or multiple pixels act as an individual detector, which is especially suitable for multi-channel application. In various embodiments, this approach can be used to measure blood flow, whether absolute, relative, or both.

DSCA can be implemented in both spatial and time domains. For spatial DSCA (sDSCA), a raw speckle image is first obtained from the sample surface. The raw speckle images may first be normalized by the smooth intensity background, which can be averaged over a number of speckle images. The speckle contrast, Kₛ is defined as the ratio of the standard deviation to the mean intensity across many detectors or pixels, Kₛ=σₛ/<I>, where subscript s refers to the spatial, as opposed to temporal, variations. The quantity Kₛ is related. to the field autocorrelation function g₁(τ) as follows:

$$V ( T ) = {[ {K}_{s} ( T ) ]}^{2} = \frac{2}{T} {\int }_{0}^{T} {( 1 - τ \text{/} T ) [ {g}_{1} ( τ ) ]}^{2} d τ$$

where V is the intensity variance across the image, and T is the image sensor exposure time. By using the known solution of the correlation diffusion equation in the semi-infinite medium, the formal relationship between the flow rate and Kₛ can be derived. The relationship between the flow and 1/Kₛ² turns out to be substantially linear in the range of flow seen in body tissue, with 1/Kₛ² increasing with increasing flow rate.

Another way to implement this speckle contrast rationale for flowmetry is to use statistical analysis on time series data obtained by integrating over a certain time. This temporal domain analysis is referred to herein as tDSCA. The integrating time for tDSCA can be regarded as analogous to the exposure time of the image sensor in sDSCA. In the case of tDSCA, a detector with moderate sensitivity with an integrating circuit can be used. For example, each pixel on a CCD chip can be used for this purpose as each CCD pixel keeps accumulating photoelectrons for a given exposure time. Therefore, a number of single-mode fibers can be directly positioned on some locations on a single CCD chip, resulting in a multi-channel tDSCA system without losing any time resolution. The number of channels is only limited by the CCD chip size, pixel size, and the area of each fiber tip. In some embodiments, tDSCA can use sensitive detectors such as avalanche photodiode (APD) and/or photomultiplier tube (PMT) with a slow counter such as a counter included in a DAQ card with USB connection, but scaling this embodiment to multichannel instrument is costly and bulky. Time-series data taken either way can be obtained by repeat measurements, for example 25 measurements can be made consecutively, after which the data can be analyzed statistically to determine the flow rate. In a configuration with an exposure time of 1 ms, one flow index would be obtained every 25 ms, resulting in approximately 40 Hz operation.

The statistical analysis of the time-series data can be substantially identical to that described above with respect to sDSCA, except that the statistics (average intensity and standard deviation of intensity) are calculated in the time domain, rather than the spatial domain. As a result, tDSCA may provide lower time resolution than sDSCA. However, the detector area for tDSCA may be significantly smaller than with sDSCA. As with the spatial domain counterpart, tDSCA provides an approach with instrumentation and analysis that are significantly simpler and less computationally intensive than traditional DCS techniques. As such, in some embodiments, a system for assessment of peripheral blood flow characteristics in deep tissue can comprise any number of the following: a support structure configured to be positioned onto a patient's anatomy; at least one diffuse optical flow sensor carried by the support structure and configured to be positioned in optical communication with a skin surface of the patient's anatomy; a coherent laser light source; a photodetector operably connected to the at least one diffuse optical flow sensor; a hardware or software processor configured to analyze data from the at least one diffuse optical flow sensor to determine absolute and/or relative blood flow at a location near the diffuse optical flow sensor when the support structure is positioned onto the patient's limb, the hardware or software processor configured to determine blood perfusion characteristics in the patient's vasculature by determining the spatial speckle contrast ratio Kₛ or the temporal speckle contrast ratio Kₜ and 1/Kₛ² or 1/Kₜ² from intensity fluctuations, and correlating 1/Kₛ² or 1/Kₜ² values with blood flow; and a feedback device configured to provide a signal indicative of the absolute and/or relative blood flow determined by the hardware or software processor. In some embodiments, the at least one diffuse optical flow sensor is configured to capture light scattered diffusively into tissue and transmitted to a depth of penetration of between, for example, about 5 mm and 50 mm, between about 5 mm and about 100 mm, between about 5 mm and about 200 mm. In some embodiments, the patient's anatomy could be a limb (such as an arm, forearm, or hand; a foot, upper, or lower leg, a torso, an abdomen, a forehead, an ear, or an internal body location including a vascular or non-vascular body lumen, or an organ for example.

Both DCS and DSCA technology can be used to evaluate on a real-time basis the absolute and/or relative blood flow in the foot, thereby providing an important tool for interventional radiologists and vascular surgeons treating ischemia in the foot. With current tools in the operating room, the physician can usually assess via X-ray fluoroscopy whether an intervention such as a balloon angioplasty procedure has succeeded in opening up and achieving patency of a limb artery. However, the clinical experience has been that structural patency as observed with fluoroscopy is not a reliable indicator of successful reperfusion of the topographical region of the foot where the ulcer wound, ischemic tissue (e.g., blackened toes) or other clinical manifestation is located. To augment fluoroscopic data on arterial patency, a plurality of DOF sensors used in either DCS or DSCA systems can be positioned at different topographical regions of the foot to assess absolute and/or relative blood flow in the different regions. For example, the topographical regions may correspond to different pedal angiosomes.

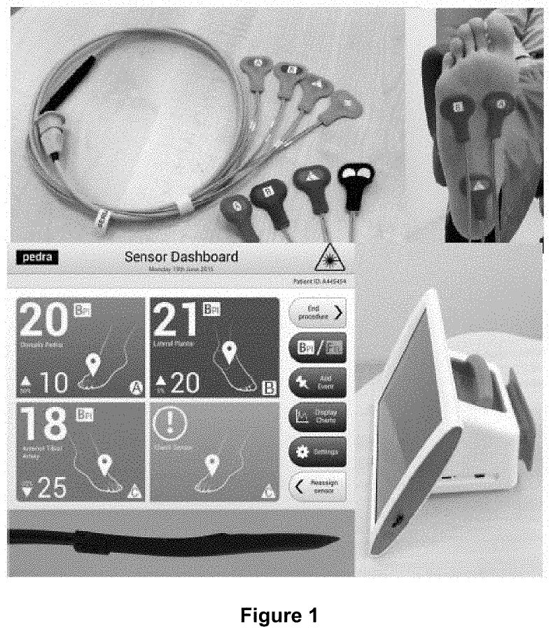

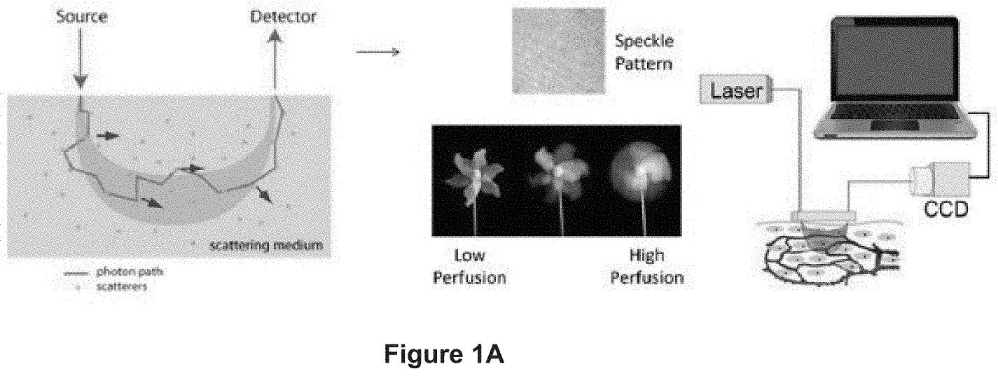

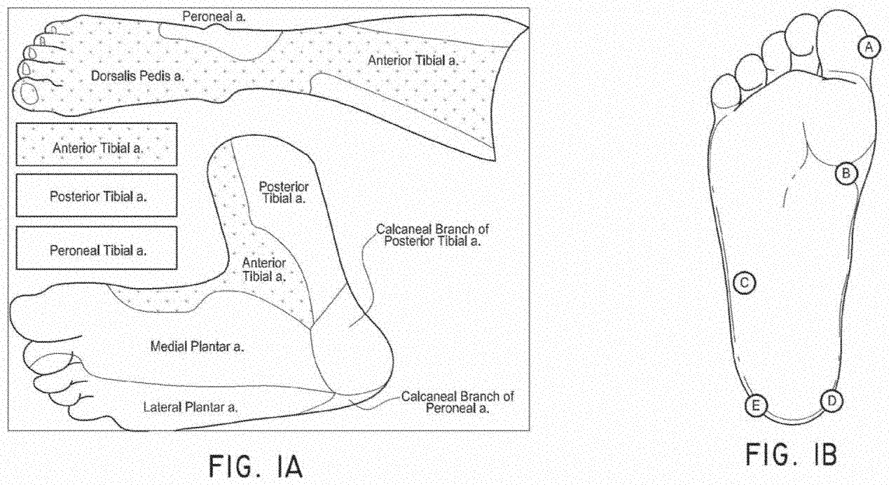

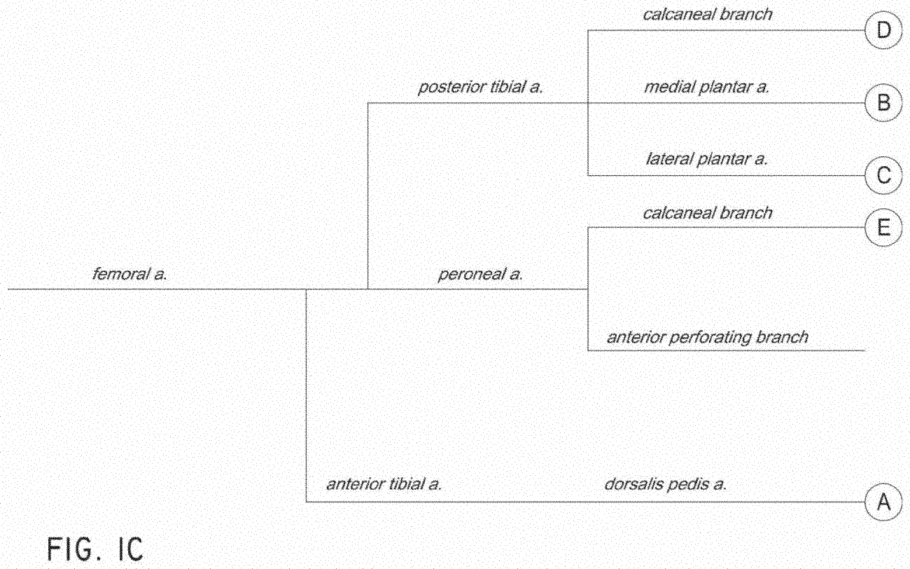

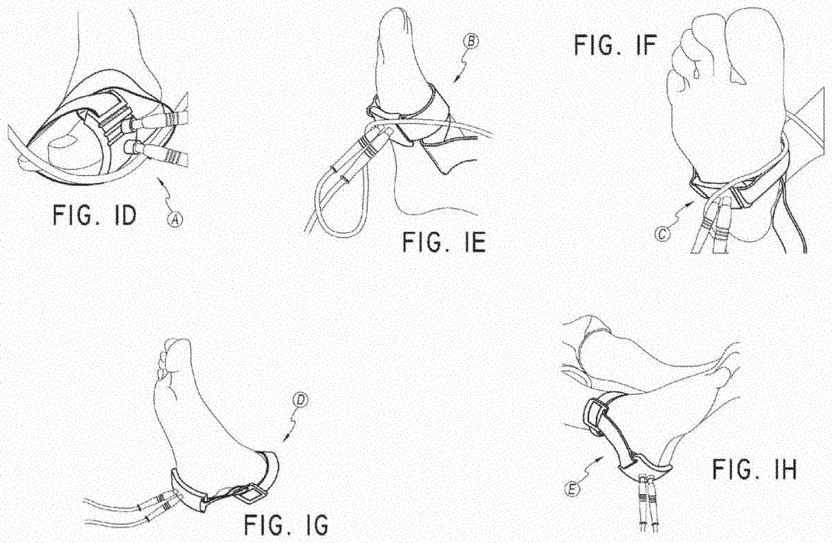

FIG. 1 illustrates a portable system comprising DSCA technology. The system can include a compact instrument console connected to a sensor that is pasted onto suitable locations on the patient's foot during perfusion assessment. In some embodiments, the device can be housed in a metal box to reduce EM emissions. No incisions are required. Via simple skin contact, the device can monitor tissue perfusion at depths of up to about, or at least about 5, 5.5, 6, 6.5, 7, 7.5, 8 mm, or more. The device can include a low flat profile sensor head that permits easy adhesive attachment to the skin. The monitor console can include opto- electronic instrumentation including coherent infrared light sources, photo detectors, and display/control electronics. The sensor can comprise passive fiber-optic conduits, which transfer infrared light from the console to the patient, and relays scattered light from the patient back to the console.

The sensors can include planar DOF sensors, which can place a fiber in optical communication with the sample. In some embodiments an optically transparent sterile barrier comprising at least one optically transparent layer may be disposed between the fiber and the sample. The at least one optically transparent layer may be configured to have adhesive coatings to facilitate attachment of the planar DOF sensor onto the surface of the sample/tissue. For example, surgical tape may comprise a support configured to receive the I)OF sensor thereon, and to couple the DOF sensor to the sample.

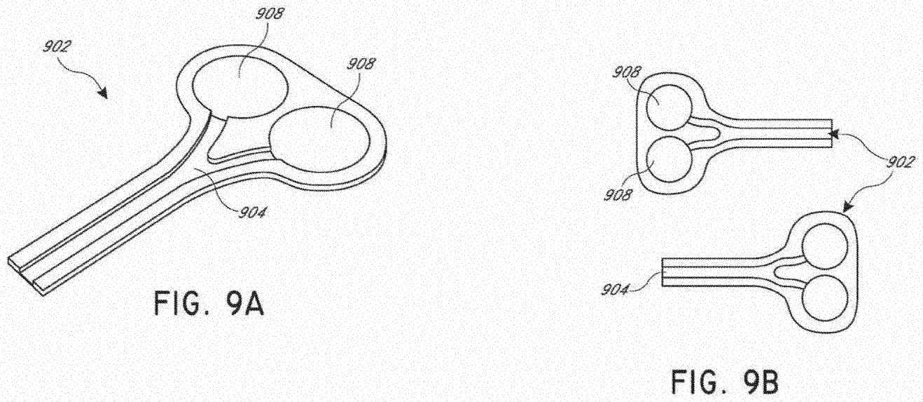

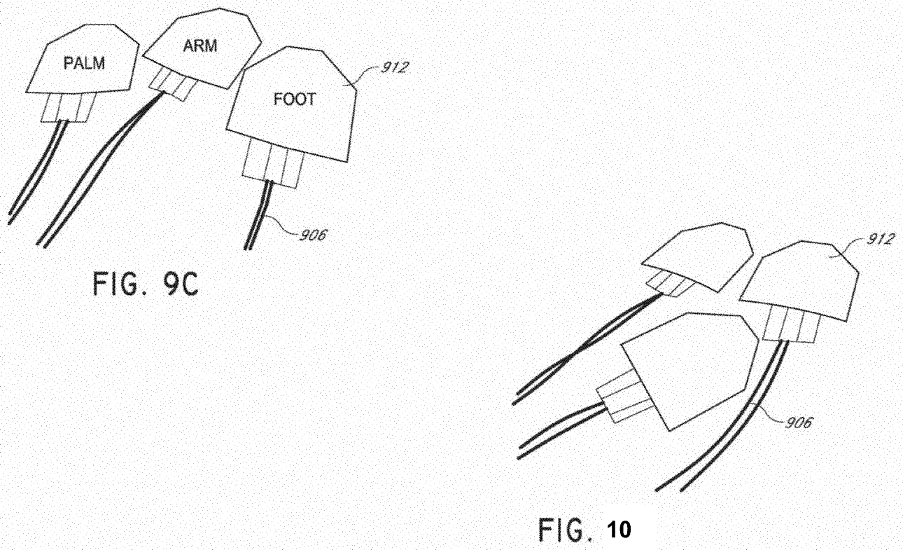

FIGS. 9A-9C show one embodiment of the supports fabricated using 3D printing, with a support comprising an adhesive layer that is disposed between the patient/tissue and the optical fibers. FIGS. 9A and 9B illustrate the support member 902, with 9C and 9D showing top and bottom views, respectively, of the sensor heads 900 prepared with a layer of surgical adhesive tape 912 to be disposed between the patient's skin and the fibers. In FIGS. 9C and 10, the reflector pads 908 and tips of fibers 906 are obscured by the adhesive liner of the surgical tape 912. In other embodiments, the at least one optically transparent layer may not have an adhesive coating, whereupon the planar DOF sensor may be attached to the sample by the application of surgical tape, a mechanical clamp, adjustable strap, or other means.

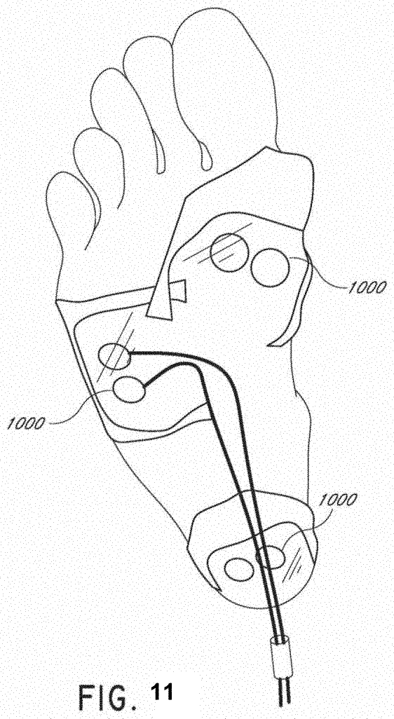

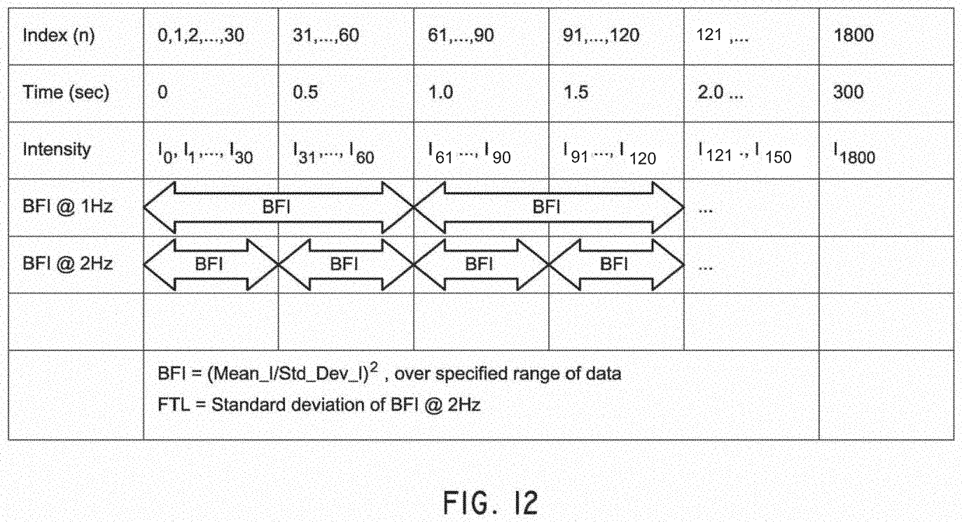

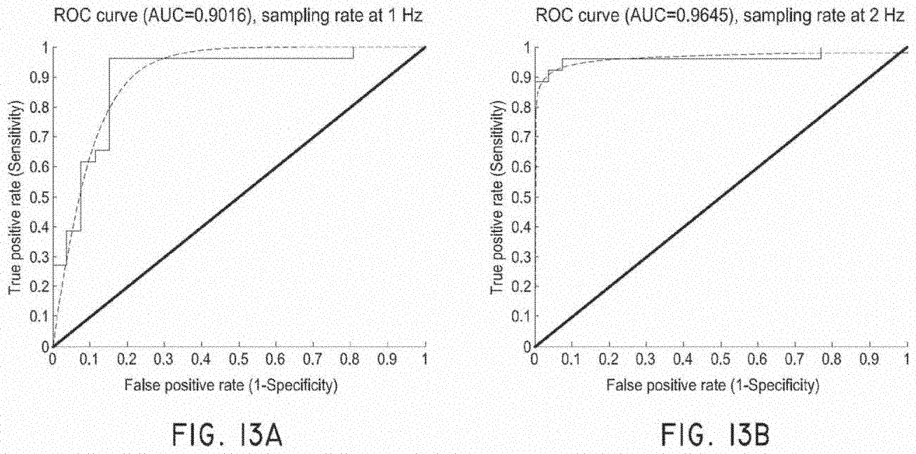

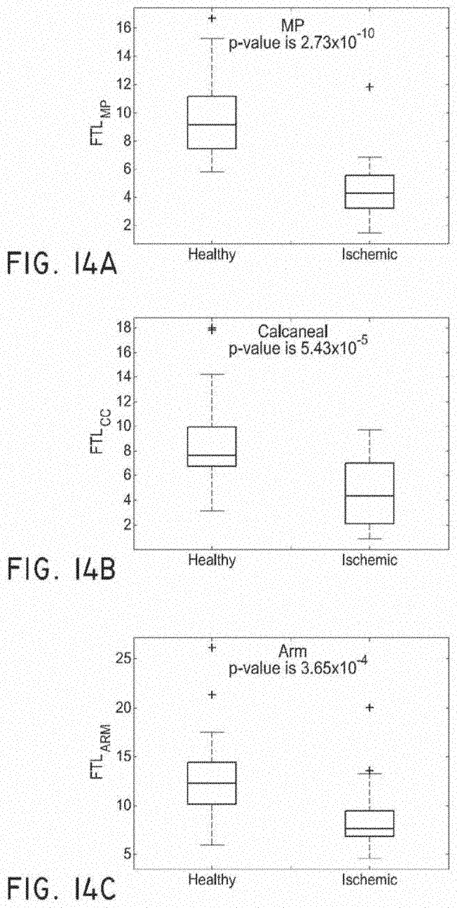

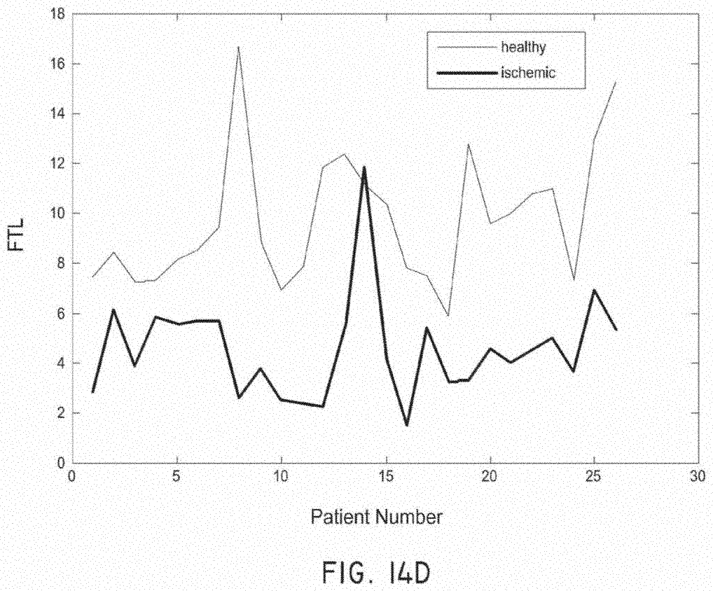

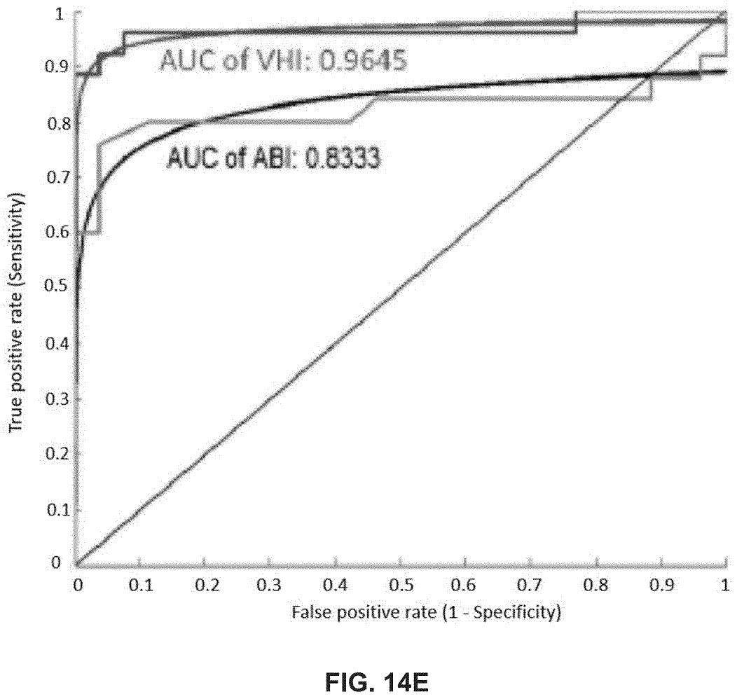

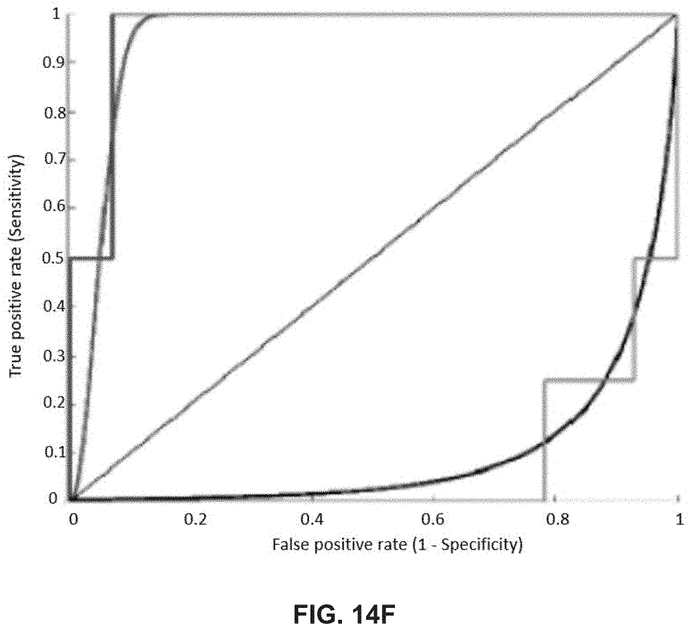

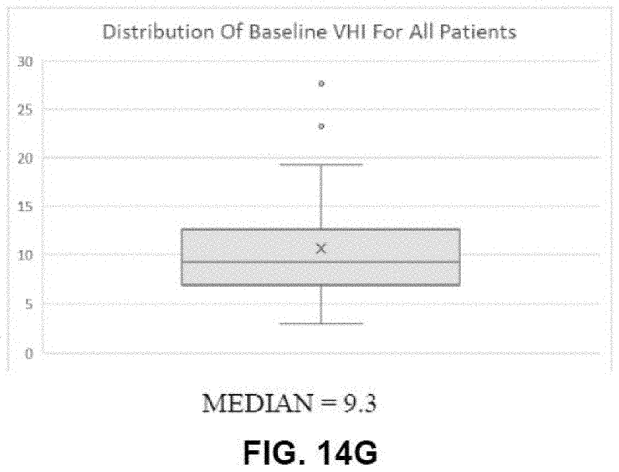

FIG. 11 illustrates a plurality of DOF sensors 1000 attached to a patient's foot. With a source-detector separation of approximately 1.5 cm on a healthy human foot, arterial cuff occlusion protocol Observations display typical blood perfusion variations—e.g., a sudden decrease and plateauing during occlusion, and sharp overshoot and subsequent recovery to baseline value after release of the cuff pressure.

An angiosome is a three-dimensional portion of tissue supplied by an artery source and drained by its accompanying veins. It can include skin, fascia, muscle, or bone. Pedal angiosomes are illustrated in FIG. 1A. Below the knee, there are three main arteries: the anterior tibial artery, the posterior tibial artery, and the peroneal artery. The posterior tibial artery gives at least three separate branches: the calcaneal artery, the medial plantar artery, and lateral plantar artery, which each supply distinct portions of the foot. The anterior tibial artery supplies the anterior ankle and continues as the dorsalis pedis artery, which supplies much of the dorsum of the foot. The calcaneal branch of the peroneal artery supplies the lateral and plantar heel. The anterior perforating branch of the peroneal artery supplies the lateral anterior upper ankle. As a result, the pedal angiosomes include: the angiosome of the medial plantar artery, the angiosome of the lateral plantar artery, the angiosome of the calcaneal branch of the posterior tibial artery, the angiosotne of the calcaneal branch of the peroneal artery, the angiosome of the dorsalis pedis artery. There is some debate as to whether there is a separate sixth pedal angiosome corresponding to the anterior perforating branch of the peroneal artery.

FIG. 1B illustrates five measurement points on the foot, each corresponding a pedal angiosome identified in FIG. 1A. By detecting blood flow in each of these positions, blood flow from the various arteries can be evaluated independently. For example, measurement of blood flow at point A (see FIG. 1D) is indicative of blood flow from the dorsalis pedis artery, and also the anterior tibial artery. Similarly, measurement of blood flow at point B (see FIG. 1E) corresponds to the medial plantar artery, while point C (see FIG. 1F) corresponds to the lateral plantar artery, point D (see FIG. 1G) corresponds to the calcaneal branch of the posterior tibial artery, and point E (see FIG. 1H) corresponds to the calcaneal branch of the peroneal artery.

FIG. 1C is a branching diagram of the arteries supplying the pedal angiosomes. The blood flow measurement points A-E are illustrated as terminating respective artery branches, though in practice the measurement points need not be at the distal-most end of the respective arteries. As noted above, measurements at any of the points A-E may provide valuable clinical information regarding local perfusion.

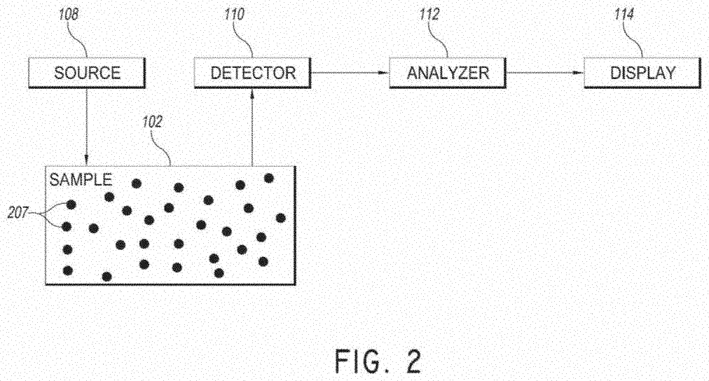

Topographical-based peripheral vascular interventions, such as angiosome-directed peripheral vascular interventions, have been developed relatively recently, and show promising performance compared with traditional intervention, particularly in terms of improved limb salvage rates. A system employing a plurality of DOF sensors can provide real-time feedback on changes in perfusion of different topographical locations in the foot, e.g. angiosome by angiosome, so that interventional radiologists or vascular surgeons may immediately evaluate whether specific intervention at a target artery has succeeded in restoring sufficient blood perfusion to the targeted topographical region of the foot where the ulcer wound, ischemic tissue or other clinical manifestation is located. FIG. 2 is a block diagram of a system for measuring flow of turbid media. A sample 102 includes a heterogeneous matrix therein. Within this matrix is an embedded flow layer with randomly ordered microcirculatory channels through which small particles 207 move in a non-ordered fashion. For example, in some embodiments the sample may be body tissue, with a complex network of peripheral arterioles and capillaries. A source 108 injects light into the sample 102. A detector 110 can detect light scattered by the moving particles 207 in the microcirculatory channels. The detector 110 can be positioned to receive light that passes from the source into the sample, and diffuses through the sample. In some embodiments, the detector can be coupled to the sample by a single-mode optical fiber. In some embodiments, the detector may be a multi-pixel image sensor, for example a CCD camera, used to image an area of the sample. In other embodiments, the detector may be a photon-counting avalanche photodiode (APD) or photomultiplier tube (PMT). As the particles flow in random direction, the scattering of light from the source 108 will vary, causing intensity fluctuations to be detected by the detector 110. An analyzer 112 is coupled to detector 110 and configured to receive a signal from the detector 110. The analyzer 112 may comprise an autocorrelator, which measures the temporal intensity autocorrelation function of light received by the detector 110. The autocorrelation function can be used to obtain the scattering and flow characteristics of the small particles flowing in the sample 102. The time-dependent intensity fluctuations reflect the time-dependent density fluctuations of the small particles 207, and accordingly the autocorrelation function can be used to determine the flow rate within the sample 102. In some embodiments, a hardware autocorrelator may be employed, while in other embodiments a software autocorrelator can be used. The flow rate or other characteristic determined by the analyzer 112 may be outputted to a display 114. The measured quantity may therefore be provided to an operator via the display 114. In various embodiments, the operator may be a clinician, diagnostician, surgeon, surgical assistant, nurse, or other medical personnel. In some embodiments, the measurement may be provided via display 114 in substantially real-time. In some embodiments, the measurement may be provided via display 114 within about 1 second from measurement, e.g., within about 1 second of the time that the scattered light is detected by the detector, the measurement may be provided via display 114. In various embodiments, the measurement may be provided within less than about 10 minutes, within less than about 5 minutes, within less than about 1 minute, within less than about 30 seconds, within less than about 10 seconds, or within less than about 1 second from detection.

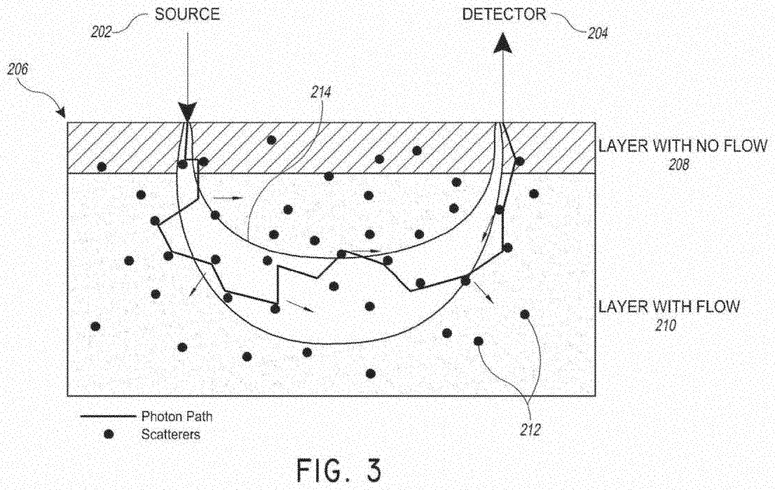

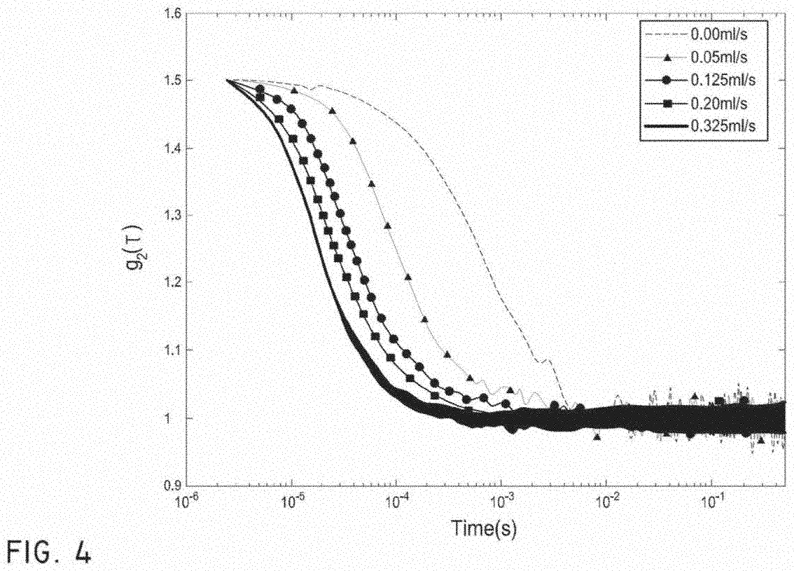

FIG. 3 is a schematic illustration of diffuse light penetration and detection in multi-layer tissue. As illustrated, a source 202 and a detector 204 are both positioned adjacent a portion of tissue 206. As noted above, in some embodiments optical fibers may be used to couple one or both of the source and detector to the tissue. The tissue 206 is multi-layer, including an upper layer 208 with no flow, and a deeper layer 210 with flow. A plurality of light-scattering particles 212 flow within capillaries in flow layer 210, and may include, for example, red blood cells. As light 214 is emitted from the source 202, it diffuses as it penetrates the tissue 206. As illustrated, a portion of the light 214 is diffused such that it is incident on the detector 204. The light 214 may follow a roughly crescent-shaped path from the source 202 to the detector 204. The depth of penetration of the light 214 detected by the detector 204 depends on the separation between the source and the detector. As the distance increases, penetration depth generally increases. In various embodiments, the separation distance may be between about 0.5 cm and about 10 cm, or in some embodiments between about 0.75 cm and about 5 cm. Preferably, in other embodiments the separation distance may be between about 1 cm and about 3 cm. In various embodiments, the separation distance may be less than about 10 cm, less than about 9 cm, less than about 8 cm, less than about 7 cm, less than about 6 cm, less than about 5 cm, less than about 4 cm, less than about 3 cm, less than about 2 cm, less than about 1 cm, less than about 0,9 cm, less than about 0.8 cm, less than about 0.7 cm, less than about 0.5 cm, less than about 0.4 cm, less than about 0.3 cm, less than about 0.2 cm, or less than about 0.1 cm. The penetration depth may vary, for example in some embodiments the penetration depth of the sensor may be between about 0.5 cm and about 5 cm, or in some embodiments between about 0.75 cm and about 3 cm. Preferably, in other embodiments the penetration depth may be between about 5 mm and about 1.5 cm. Of course, the tissue optical properties of the various layers also contribute to the penetration depth of the light, as does the intensity, wavelength, or other characteristics of the light source. These variations can allow for the depth of measurement to be adjusted based on the part of the body being analyzed, the particular patient, or other considerations. Measurements obtained by the detector 204 may then be processed and analyzed to calculate the autocorrelation function. As seen in FIG. 4, the autocorrelation function may be used to determine the flow rate in the tissue.

FIG. 4 is a graph of autocorrelation functions for different flow rates, with steeper decay of the autocorrelation curve indicating faster flow rates. The autocorrelation curves are plotted on a semi-logarithmic scale in the graph. As is generally known in the art, blood flow data can be analyzed by fitting each autocorrelation curve to a model, such a semi-infinite, multi-layer diffusion model, The fitted autocorrelation curves can then provide relative blood flow rates, which can be usefully applied during peripheral interventional procedures such as balloon angioplasty or surgery, or as a diagnostic tool. In some embodiments, systems and methods are configured such that no autocorrelation is utilized.

Diffuse optical flow (DOF) sensors (which, as described above, can include either or both DCS and DSCA sensors) can be particularly useful in measuring microcirculation, for example in measuring blood perfusion in the foot. This technique can be additionally improved by employing the concept of pedal topography. One example of a topographical analysis of blood flow in the foot incorporates the concept of pedal angiosomes, as described above,

Systems and methods can involve DSCA to calculate two quantitative indices: (a) Blood Perfusion Index (“BPI”), also referred to as Blood Flow Index (“BFI”) elsewhere herein; and (b) Vascular Health Index (“VHI”), also referred to as Low Frequency Oscillation Index (“LFI”) elsewhere herein. BPI reflects real-time tissue perfusion as measured on a quantitative scale, while VHI is a derivative index generated from 5 minutes of raw BPI data subjected to an algorithm described elsewhere herein.

A diagrammatic representation of the DSCA process is shown in FIG. 1AA. DSCA works on the principle that as coherent light propagates from the source fiber through the patient's tissue, it is scattered by blood cells. The light signal collected at the detector fiber is an agglomeration of photons that have traversed the patient's tissue via a multitude of different scattering trajectories. At any given instant in time, the average path length of the photons arriving at the detector fiber will determine if these photons will interfere (with each other) in a constructive or destructive fashion. The net result is that the detected light intensity fluctuates or flickers over time, and the rate at which this occurs is indicative of the number and speed of blood cells in the patient's tissue. in summary—the higher the flicker rate of the detected signal, the higher the perfusion/BPI of the patient.

Older technologies such as laser Doppler and speckle imaging have been in existence for decades. These analyze photons that undergo a single scattering event, which consequently limits their ability to assess perfusion beyond skin depth. In contrast, systems and methods as disclosed herein can utilize an advantageous optical system to analyze photons across multiple scattering events. This can permit tissue perfusion measurements up to depths roughly 10 times greater than with either laser Doppler or speckle imaging.

Another advantage of systems and methods as disclosed herein is, unlike Doppler ultrasound and pulse oximetry, its technology is not dependent on pulsatile blood flow. These older technologies were designed to be used only on larger blood vessels larger than 1.1 mm; their optical systems cannot detect/analyze the movement of blood cells in microvascular/capillary tissue beds. This distinction can be clinically important, and is one of the reasons why ABI, which relies on Doppler ultrasound, often cannot be detected in diabetic patients who lack pulsatile flow in their feet.

DSCA systems and methods as disclosed herein can advantageously pick up relatively small changes in vessel flow. One example of this is the small but noticeable increase in BPI when an angiogram is performed. As the contrast bolus forces blood cells through the capillaries at an increased rate, the BPI spikes up briefly, and then falls as the contrast (devoid of blood cells) passes through the foot capillary bed. A second example is a drop in BPI as the IR team feels for, and compress, the distal foot pulses after the procedure.

Systems and methods as disclosed herein can provide almost instantaneous feedback on their interventions. While some procedures have a clear and obvious benefit in terms of improving blood flow and perfusion, the clinicians' experience has been that in an increasing number of patients with complex distal disease patterns, especially those with diabetic lesions, it can be difficult to ascertain the success of a procedure by angiographic guidance alone. As such, the inventive systems and methods can provide useful guidance about when to perform additional intervention and when the IR has achieved enough. Furthermore, systems and methods can have a negligible impact on workflow, and the radiolucent sensors do not interfere with foot imaging. Additional advantages are that the disclosed systems and methods are much easier to use compared to the transcutaneous tissue oxygen monitor (TcPO₂). The transcutaneous oxygen monitor was assessed to be not very robust and time-consuming, e.g., the device needed the patient to be still for 20 minutes whilst recording. As such, the disclosed systems and methods can provide a much faster and simpler measurement protocol.

Furthermore, some embodiments include a simple box design and sensors, and there is no need for pre-measurement calibration. The transcutaneous monitor requires several minutes to calibrate prior to each use, and the sensors require active monitoring during assessment as any aberrant readings will require re-calibration of sensors to ensure adequate results. The fixation devices for the TcPO₂ sensors are not always adherent to skin; sometimes the adhesive wore off and monitoring had to restart, which further prolonged the measurement time. There is a cost aspect as well; the transcutaneous oxygen monitor requires the purchase of membranes and fixation probes which must be purchased regularly. Any failure to use the membranes and fixation probes results in inaccurate values.

The assessment of ABIs and TBIs reflected mainstream clinical opinion as being unreliable and prone to falsely elevated values due to non-compressible vessels. A significant number of patients were unable to undergo these procedures as they found the cuff pain intolerable. In contrast, systems and methods as disclosed herein can be pain-free and has well tolerated by all patients, and not result in skin damage or irritation caused by the use of the device and sensors.

In many cases, prior to vascular intervention, an interventional radiologist or vascular surgeon will image the vasculature of interest, for example using fluoroscopy, computed tomography, ultrasound, or other imaging technique. With such imaging, several potential occlusions or lesions may be identified, Peripheral intervention, such as balloon angioplasty, atherectomy, or surgical bypass/grafts can be employed to re-open one or more of the identified occlusions or lesions (“the target lesions”), in an effort to restore perfusion to the affected region(s) of the foot. For these peripheral interventions to result in successful limb salvage, blood perfusion must reach a sufficient level that permits healing of the foot wound. Without a real-time perfusion monitor, a physician has no way of knowing for sure if an intervention has achieved an improvement in perfusion sufficient for wound healing, or at all. The use of real-time measurement of blood perfusion at various topographic locations of the foot, as described herein, addresses this problem. It provides objective quantitative perfusion data in real-time so that the physician can know with certainty whether a specific intervention at a target lesion has succeeded in restoring perfusion to the topographic region of the foot on which the wound is located. If a determination has been made that an acceptable level of perfusion at the desired topographic region has been achieved, the physician can avoid the additional risk associated with further intervention, and bring the procedure to a close. Alternatively, if a specific intervention at a target lesion has not resulted in any perfusion improvement as measured by a real-time perfusion monitor, the physician will thereby be guided to undertake the additional risk of proceeding onto secondary target lesions. The use of a real-time perfusion monitor thus averts the situation where a peripheral intervention procedure is ended prematurely prior to achieving the desired improvement in perfusion. It also guides physicians as to which target lesion (when revascularized) resulted in the greatest perfusion improvement at the desired topographic region of the foot. This real-time knowledge would in turn inform the physician as to the optimal placement for use of a drug-eluting balloon or other means to prolong the patency of the vessel in which the said lesion is located.

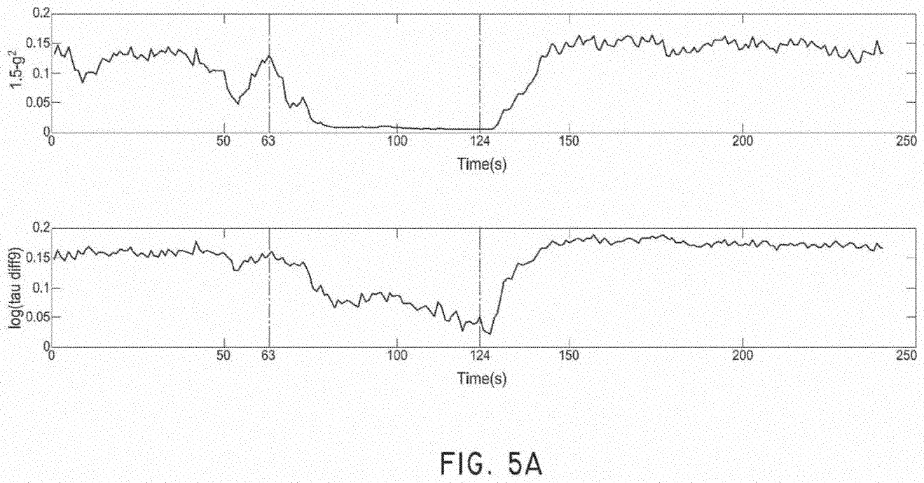

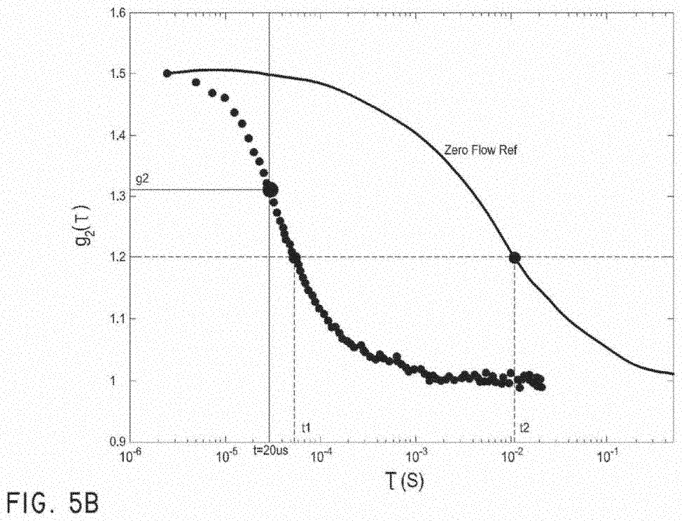

Although changes in perfusion can be seen directly from the change in shape of the autocorrelation function, potentially more useful ways to define a blood flow index (BFI), which may also be referred to herein as a blood perfusion index (BPI) have been developed. FIG. 5A is a graph of two such BM over time during a cuff occlusion protocol. The dashed vertical lines indicate the starting and stopping times of the cuff inflation, The top chart illustrates a BFI calculated from vertical crossing of the autocorrelation curve, while the lower chart illustrates a BFI calculated from horizontal crossing of the autocorrelation curve. FIG. 5B is a graph illustrating these two different methods of calculating BFI. The solid line represents the zero flow reference data, while the dotted line represents real-time autocorrelation data. The vertical crossing indicator compares the y-axis value (g₂) of the real-time autocorrelation data and the reference data at a given time. For example, the first indicator can be calculated as 1/g₂ or 1.5-g₂. The horizontal crossing indicator compares the time difference between the autocorrelation data and the reference data at a given flow rate. For example, the second indicator can be calculated as log(t2/t1).

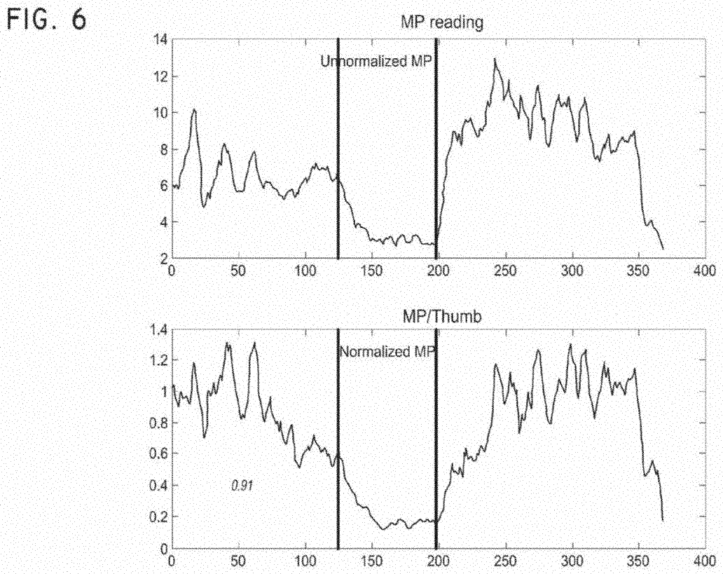

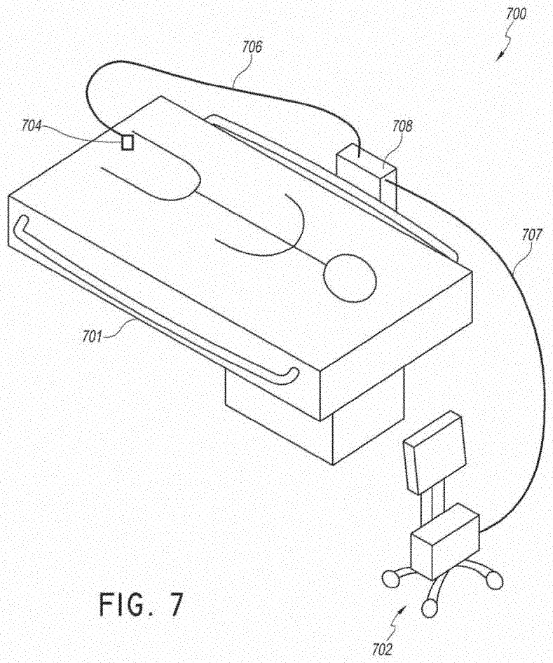

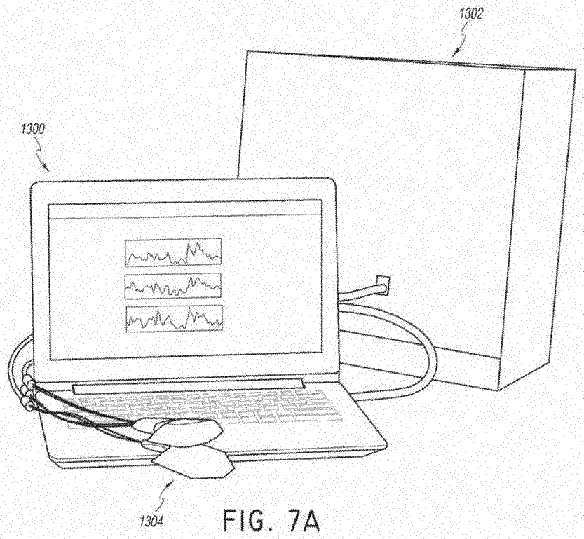

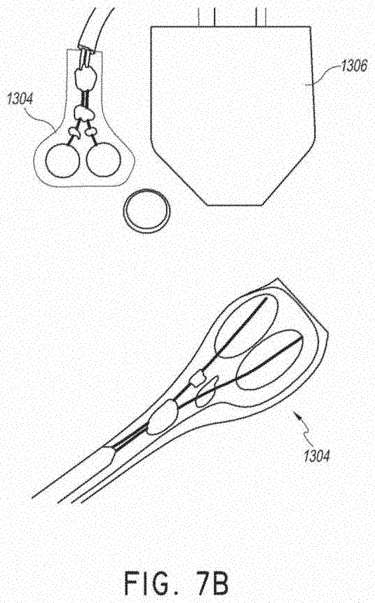

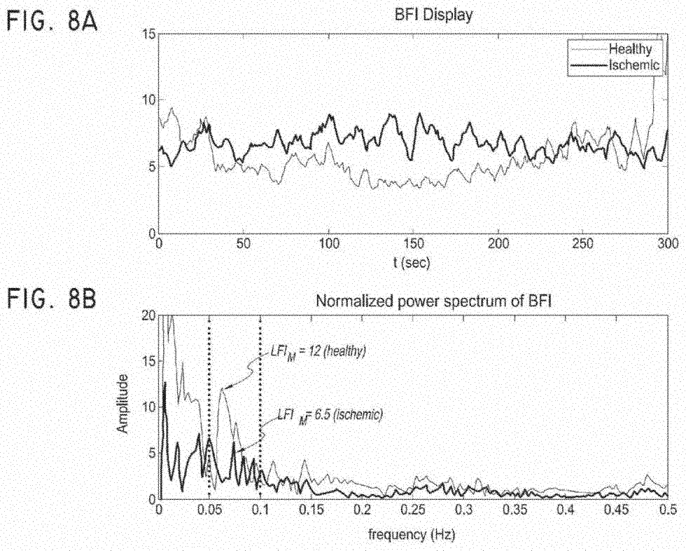

Charts such as those shown in FIG. 5A, or other such indicia of blood flow, can be displayed to an operator in real-time via audible, visual, or tactile feedback. A physician may thereby be provided with substantially real-time feedback on the efficacy of a peripheral intervention. For example, during balloon angioplasty, a physician can monitor the BFI as measured on a specific location of the foot. The BFI will decrease while the balloon is inflated, and increase after deflation. After repeated inflation of the balloon to perform the angioplasty, the BFI should increase relative to the pre-angioplasty baseline, indicating that the angioplasty procedure has resulted in an improvement in perfusion at the target foot tissue. A BFI that does not increase relative to the pre-angioplasty baseline indicates that the balloon angioplasty was not successful in restoring perfusion. Providing such feedback in real-time is an enormous benefit to physicians performing vascular intervention. Rather than waiting post-operatively for hours or days to determine whether perfusion has been improved, during which time the foot may deteriorate to the point of requiring amputation, the use of DOF sensors at select pedal locations during the angioplasty procedure can provide immediate feedback, allowing the physician to continue, modify, or conclude the procedure as needed. As noted above, in various embodiments, the feedback may be provided, in some cases, within less than about 10 minutes, within less than about 5 minutes, within less than about 1 minute, within less than about 30 seconds, within less than about 10 seconds, or within less than about 1 second from measurement. In some embodiments, success of a revascularization procedure can be indicated by an increase in BFI of about or at least about 5%, 10%, 15%, 20%, 25%, 30%, 35%, 40%, 45%, 50%, 55%, 60%, 65%, 70%, 75%, 80%, or more compared to a BFI value prior to the procedure. FIG. 6 is a graph of two BFIs during cuff occlusion protocol. FIG. 7 illustrates various elements of a perfusion monitoring system, according to some embodiments. FIG. 7A illustrates an embodiment of a DSCA perfusion monitor console and instrumentation box. FIG. 7B illustrates embodiments of low-profile sensors. FIG. 8A shows the raw BFI data (raw time series BFI data) measured at the medial plantar section of the foot of two individuals, one healthy versus one with indications of limb ischemia, while FIG. 8B shows the equivalent power spectrum data of the same individuals (Fourier transform of raw time series BFI data.

While the example above relates to balloon angioplasty, the use of DOF sensors to assess blood flow (whether relative, absolute, or both) in the foot can be advantageously applied before, during, or after a number of different interventions. For example, DOF sensors can be used to aid interventions such as rotational atherectomy, delivery of lytic substances including but not limited to tPA, bypass procedures, stent and/or graft placement, or any other intervention.

In some embodiments, systems can include a hardware or software processor configured to receive signals and calculate absolute BPI values and/or the BPI ratio, which can be defined as the BPI at a second, later point in time (e.g., the end of a revascularization procedure, for example), divided by the BPI at a first, earlier point in time (e.g., a pre-procedure baseline, the start of a revascularization procedure, or an intraoperative baseline, for example). The intraoperative baseline in some cases can be just prior to, such as within 5, 4, 3, 2, 1 minute, 30 seconds, 15 seconds, 10 seconds or less with respect to the first angioplasty of a vascular procedure. The intraoperative baseline can be in some cases after the guidewires have been placed and before the first ballooning or other intervention has taken place. In some embodiments, the intraoperative baseline can be taken automatically upon activation of a control, and then automatically expand the balloon and/or take another action with respect to the procedure within a predetermined time after taking the intraoperative baseline. In some embodiments, the system can be configured to automatically take a second measurement can take place at a predetermined time following deflation of the balloon for example.

In some embodiments, a BPI index need not necessarily be a ratio, but can be calculated by the BPI at a second, later point in time minus the BPI at a first, earlier point in time for example.

In some embodiments, a BPI index (such as a ratio, subtraction of two values, or other index) can involve two or more values separated in time by about, at least about, or no more than about 5 minutes, 10 minutes, 20 minutes, 30 minutes, 40 minutes, 50 minutes, 60 minutes, 90 minutes, 120 minutes, 150 minutes, 180 minutes, 4 hours, 5 hours, 6 hours, 8 hours, 10 hours, 12 hours, 16 hours, 24 hours, 36 hours, 2 days, 3 days, 4 days, 5 day, 6 days, 7 days, 2 weeks, 3 weeks, 1 month, 2 months, 3 months, 4 months, 5 months, 6 months, or more or less including ranges involving two or more of the preceding values.

In some embodiments, a BPI Ratio can be compared against the % change in discharge BPI (e.g., taken within 36 hours, and often at around 12 hours after the procedure) relative to the pre-op baseline BPI (e.g., taken in clinic at patient admission or shortly before the procedure).

In some embodiments, a BPI ratio, including but not limited to an intra-op BPI Ratio, of greater than about 1, 1.1, 1.2, 1.25, 1.3, 1.4, 1.5, 1.6, 1.7, 1.75, 1.8, 1.9, 2, 2.5, 3, or more, or ranges including any two of the foregoing values may have the potential to predict a positive % perfusion change resulting from the procedure, and also be predictive of positive patient outcomes. Table 1, including data from a study of such potentially predictive values, is shown below. The converse is true in that a BPI Ratio less than about 1, 0.9, 0.8, 0.7, 0.6, 0.5, or less in one or more sensors on the foot can be predictive of a flat/negative change in % BPI and mixed/negative patient outcomes. Not to be limited by theory, but relatively small determined changes in % BPI (or VIE), among other indices has surprisingly and unexpectedly found to correlate with patient outcomes. As such, some embodiments of systems and methods can be used to predict a response to a completed intervention, and/or determine in real-time while the patient is on-table whether a sufficiently satisfactory result has been achieved, or conversely that more intervention steps are required. The same or other embodiments can be used to assess if the completed intervention is likely or unlikely to have a successfully clinical effect, or provide a pre-intervention recommendation and/or prediction related to the patient's clinical condition. In some embodiments, systems and methods can communicate a recommendation and/or prediction to an operator or third party based on the BPI Ratio, % BPI, VHI, or other indices via a display, audio communication, and the like. In some embodiments, the system or method can electronically display the numerical ratio and/or a shaded or colored bar or other metric of the numerical ratio. For example, a ratio of less than a predetermined threshold value, such as a BPI ratio of 1 as one example, may be indicated in a first color, such as red, while a BPI ratio of greater than a predetermined threshold value, such as a BPI ratio of 1 for example, may be indicated in a second color, such as green. For example, as illustrated in Table 1, in a study of 35 patients each of whom had 2 sensors on the foot, the use of a BPI Ratio>1 (with BPI Ratio>1 predicting positive outcome and BPI Ratio<1 predicting negative outcome, with the exception of very low BPI pre-op baselines which required a higher BPI Ratio) demonstrated a 91% correlation with clinical outcome. Within the study, this compared favorably with a correlation of only 33% based on an assessment of a change in TcP02; 52% based on an assessment of a change in ABI; 67% based on an assessment of a change in TBI; and 77% based on an assessment of the change in angiographic markers (typically considered the gold standard).

TABLE 1
Correlation of BPI Ratio to Patient Outcome

| | | | Patient | | | | BPI Ratio | |
|---|---|---|---|---|---|---|---|---|
| Patient | | | Outcome | Pre- | Post- | BPI | Consistent | |
| ID | Channel | Group | at Discharge | BPI | BPI | Ratio | w Outcome? | Continents |
| 1001 | A | CLI | Good | | | NA | NA | |
| 1001 | B | | | | | NA | NA | |
| 1002 | A | CLI | Good | 27.5 | 32.5 | 1.2 | Yes | |
| 1002 | B | | | 21.5 | 22.5 | 1.0 | Yes | |
| 1003 | A | PAD | Good | 13.5 | 45 | 3.3 | Yes | |
| 1003 | B | | | 22.5 | 52.5 | 2.3 | Yes | |
| 1004 | A | CLI | Short-term | 6.5 | 15 | 2.3 | Yes | BPI Ratio predicted short-term outcome. |
| | | | Good | | | | | |
| 1004 | B | | | 10 | 15 | 1.5 | Yes | |
| 1005 | A | CLI | Poor | NA | NA | NA | NA | Real-time BPI chart not collected due to |
| | | | | | | | | console freezing. |
| 1005 | B | | | NA | NA | NA | NA | |
| 1006 | A | PAD | Flat/Poor | 15 | 17 | 1.1 | No | |
| 1006 | B | | | 17 | 16 | 0.9 | Yes | |
| 1007 | A | PAD | Flat/Good | 9 | 6 | N/A | N/A | Not possible to get BPI Ratio as BPI signal |
| 1007 | B | | | 9 | 7 | N/A | N/A | dampened by large sheath; no angiographic |
| | | | | | | | | images. |
| 1008 | A | CLI | Poor | 27 | 25 | 0.9 | Yes | |
| 1008 | B | | | 32.5 | 24 | 0.7 | Yes | |
| 1009 | A | CLI | Poor | 10 | 12 | 1.2 | Yes | A BPI Ratio of 1.2 is not high enough given |
| | | | | | | | | the low base of perfusion. |
| 1009 | B | | | 26 | 18 | 0.7 | Yes | |
| 1010 | A | PAD | Good | 12 | 19 | 1.6 | Yes | |
| 1010 | B | | | 7.5 | 10 | 1.3 | Yes | |
| 1011 | A | CLI | Flat/Good | 23 | 21.5 | 0.9 | No | Lower BPI Ratio may be due to downward |
| | | | | | | | | drift of laser intensity. |
| 1011 | B | | | 28 | 28.5 | 1.0 | Yes | |
| 1017 | A | CLI | Flat/Poor | 14 | 12 | 0.9 | Yes | |
| 1017 | B | | | 14 | 13 | 0.9 | Yes | |
| 1018 | A | PAD | Good | 16 | 15 | 0.9 | No | BPI Ratios likely dampened by CO₂ |
| | | | | | | | | angiography. |
| 1018 | B | | | 12 | 14 | 1.2 | Yes | |
| 1019 | A | PAD | Flat/Good | 4.5 | 4 | N/A | N/A | The damped signal due to the unoticed |
| 1019 | B | | | 7.5 | 5 | N/A | N/A | proximal SFA stenosis precludes the reliable |
| | | | | | | | | generation of BPI ratio. |
| 1020 | A | CLI | Flat/Good | 12 | 13 | 1.1 | Yes | |
| 1020 | B | | | 53 | 48 | 0.9 | No | The BPI Ratio may be artificially lowered by |
| | | | | | | | | use of larger sheath. |
| 1021 | A | CLI | Flat/Good | 24 | 23 | 1.0 | Yes | Possible dampening by CO₂ angiography. |
| 1021 | B | | | 22 | 23 | 1.0 | Yes | Possible dampening by CO₂ angiography. |
| 1022 | A | PAD | Good | 8.5 | 13 | 1.5 | Yes | |
| 1022 | B | | | 7 | 12 | 1.7 | Yes | |
| 1023 | A | PAD | Good | 9 | 11 | 1.2 | Yes | |
| 1023 | B | | | 28 | 36 | 1.3 | Yes | |
| 1024 | A | PAD | Flat | NA | NA | NA | NA | |
| 1024 | B | | | NA | NA | NA | NA | |
| 1025 | A | CLI | Flat | 4 | 5 | 1.3 | Yes | The BPI Ratio >1 are not good enough given |
| 1025 | B | | | 5 | 6 | 1.2 | Yes | the low baseline perfusion. The raw signal |
| | | | | | | | | intensity of the BPI signal fell to <15,000 |
| | | | | | | | | relative to mean intensity of 20,000 and above. |
| 1034 | A | CLI | Poor | 27 | 12.5 | 0.5 | Yes | |
| 1034 | B | | | 19 | 6 | 0.3 | Yes | |
| 1035 | A | PAD | Good | 4.5 | 6.5 | 1.4 | Yes | |
| 1035 | B | | | 4.7 | 8 | 1.7 | Yes | |
| 1036 | A | PAD | Flat | 5.5 | 6 | 1.1 | Yes | BPI Ratio of 1.1 insufficient given low |
| 1036 | B | | | 10 | 9 | 0.9 | Yes | baseline perfusion. Also taking into |
| | | | | | | | | consideration both channels. |
| 1037 | A | CLI | Flat/Good | 11.5 | 14.5 | 1.3 | Yes | |
| 1037 | B | | | 17 | 23 | 1.4 | Yes | |
| 1038 | A | PAD | Flat/Good | 19.5 | 32 | 1.6 | Yes | |
| 1038 | B | | | 15 | 18.5 | 1.2 | Yes | |
| 1039 | A | CLI | Flat/Good | 19 | 23 | 1.2 | Yes | Consistent with initial improvement at |
| | | | | | | | | discharge. |
| 1039 | B | | | 8 | 8.5 | 1.1 | Yes | |
| 1040 | A | CLI | Flat/Poor | 11 | 7.5 | 0.7 | Yes | |
| 1040 | B | | | 3.5 | 3 | 0.9 | Yes | |
| 1041 | A | CLI | Flat/Good | 12 | 16.5 | 1.4 | Yes | The BPI ratio was consistent with the |
| 1041 | B | | | 9 | 11 | 1.2 | Yes | improvement seen at discharge, but not at 30 d. |
| | | | | | | | | The pedra readings at 30 d were consistent |
| | | | | | | | | though with the re-occlusion of vessels seen on |
| | | | | | | | | the 30 d duplex. |
| 1042 | A | CLI | Flat/Poor | 25 | 7 | 0.3 | Yes | |
| 1042 | B | | | 7 | 11 | 1.6 | No | |
| 1043 | A | PAD | No | NA | NA | NA | NA | |
| | | | intervention | | | | | |
| 1043 | B | | | NA | NA | NA | NA | |
| 1044 | A | PAD | No | NA | NA | NA | NA | |
| | | | intervention | | | | | |
| 1044 | B | | | NA | NA | NA | NA | |
| 1045 | A | PAD | Flat/Good | 8 | 10 | 1.3 | Yes | |
| 1045 | B | | | 12 | 12 | 1.0 | Yes | |
| 1046 | A | CLI | Good | 11 | 15.5 | 1.4 | Yes | |
| 1046 | B | | | 8.5 | 16.5 | 1.9 | Yes | |
| 1047 | A | PAD | Flat/Good | 15 | 19 | 1.3 | Yes | |
| 1047 | B | | | 4.5 | 7.5 | 1.7 | Yes | |
| 1048 | A | PAD | Flat/Good | 9 | 10 | 1.1 | Yes | |
| 1048 | B | | | 7 | 8 | 1.1 | Yes | |
| | | | | | | | BPI Ratio | |

| | | |
|---|---|---|
| Total No of Measurements | 56 | |
| Consistent with outcome | 51 | 91% |
| Inconsistent with outcome | 5 | 9% |

A potential caveat may apply in the cases where the patient's pre-plasty absolute BPI is unusually low, e.g., less than about 20, 15, 10, 5, or even less. In such cases, it is possible that a BPI Ratio of greater than about 2 or other values as described herein may serve as a more appropriate minimum perfusion target, and/or may be required for predictive value given the low values of pre-PTA BPI.

Not to be limited by theory, use of the BPI ratio, for example, of greater than 1 or more as a real-time perfusion target for revascularization can greatly assist physicians in their real-time decision-making. For example, if a BPI Ratio less than about 1 is seen after opening a more accessible target lesion, especially if displayed by the sensor located on the wound angiosome, that should prompt consideration of more aggressive treatment to open the more difficult target lesions. This can advantageously avoid costly readmissions and/or excessive tissue loss.

In some cases, the smoothing algorithm of the BPI signal can cause a slight lag of about one minute in reflecting real-time perfusion changes; shorter if the change is significant. It may be useful in some cases to wait for a period of time, such as about 30 seconds, 1 minute, 2 minutes, or more, before measuring a BPI and calling an end to the procedure too quickly after a final plasty.

In some embodiments, the BPI signal at the distal foot can be obscured or become less sensitive when proximal flow is impeded, caused by issues including, for example, a contralateral iliac sheath, multiple SFA stenoses which were treated only later in the procedure, as well as a very tight SFA stenosis which was missed and left untreated, the introduction of a larger sheath, or the passage of catheters and wires through the very tight vein graft stenoses. As such, in some cases, it may be beneficial to address proximal lesions first, and also potentially take an intra-op baseline only after the placement of wires, before the first plasty is attempted.

In addition to the BPI Ratio analysis described above, the absolute values of pre-op and discharge BPI, and/or the percentage change of the latter over the former, may be useful in determining the a wound-healing perfusion threshold or other clinical metric as described for example herein. Table 2 below illustrates a selection of cases where there was a dearly positive outcome, and correlation with changes in BPI.

TABLE 2

| | | | | | | | | % BPI |
|---|---|---|---|---|---|---|---|---|
| Case | Group | Positive Outcome | Diabetes | Enrolment BPI | | Discharge BPI | | Change |
| 1002 | CLI | Healing of large ulcer | No | 8 | (Channel A) | 33 | (Channel A) | 312.5% |
| 1003 | PAD | Elimination of pain | Yes | 17 | (Sensor A) | 70 | (Sensor A) | 312% |
| | | | | 22 | (Sensor B) | 40 | (Sensor B) | 81% |
| 1010 | PAD | Elimination of pain | Yes | 13 | (Sensor A) | 46 | (Sensor A) | 254% |
| | | | | 12 | (Sensor B) | 38 | (Sensor B) | 216% |
| 1023 | PAD | Elimination of pain; | Yes | 8 | (Sensor A) | 25 | (Sensor A) | 212.5% |
| | | ulcer getting smaller | | 13 | (Sensor B) | 15 rising | (Sensor B) | 15-169% |
| | | | | | | to 35 | | |
| 1035 | PAD | Significant reduction | No | 6 | (Channel A) | 28 | (Sensor A) | 366% |
| | | of pain; much longer | | 7 | (Channel B) | 37 | (Channel B) | 428% |
| | | walking distance. | | | | | | |

As such, an absolute and/or percentage increase in BPI values, determined by a software or hardware processor and output to a display, for example, can guide towards a prediction of procedural success and/or wound healing. For instance, a perfusion level of at least about 20, 25, 30, 35, or more BPI may be adequate for positive wound healing in a non-diabetic patient, but higher perfusion, such as a BPI of at least about 30, 35, 40, 45, 50, or more may be needed for diabetic wound healing.

In some embodiments, systems and methods can provide outpatient diagnosis of ischemia that warrants intervention. For example, a processor can be configured to yield a second index called VHI (Vascular Health Index, also referred to as FTL herein) which in some cases can advantageously yield a better diagnostic curve for outpatient use because it is obtained via an algorithm that analyzes the perfusion fluctuations of a 5-minute log of raw BPI data (e.g., sampled at 0.5 Hz, 1 Hz, 1,5 Hz, 2 Hz, 2.5 Hz, 3 Hz or other frequencies). By its nature, it can be a more stable, computationally-derived index, less impacted by the extremes of real-time BPI signal variations reflecting second-by-second changes in physiological perfusion.

In some embodiments, the absolute values of pre-op and discharge VHI, and/or the percentage change of the latter over the former (e.g., the VHI Ratio), may be useful in determining the wound-healing perfusion threshold. Table 3 below illustrates a selection of cases where there was a clearly positive outcome, and correlation with changes in VHI.

TABLE 3

| | | | | Enrollment | | Discharge | | % VHI |
|---|---|---|---|---|---|---|---|---|
| Case | Group | Positive Outcome | Diabetes | VHI | | VHI | | Change |
| 1002 | CLI | Healing of large ulcer | No | 3.72 | (Ch A) | 11.56 | (Ch A) | 210% |
| 1003 | PAD | Elimination of pain | Yes | 7.71 | (Sensor A) | 19.24 | (Sensor A) | 150% |
| | | | | 9.3 | (Sensor B) | 17.76 | (Sensor B) | 91% |
| 1010 | PAD | Elimination of pain | Yes | 6.78 | (Sensor A) | 16.27 | (Sensor A) | 140% |
| | | | | 7.19 | (Sensor B) | 12.86 | (Sensor B) | 79% |
| 1023 | PAD | Elimination of pain; | Yes | 4.62 | (Sensor A) | 10.4 | (Sensor A) | 206% |
| | | ulcer getting smaller | | 5.48 | (Sensor B) | 16.78 | (Sensor B) | |
| 1035 | PAD | Significant reduction | No | 2.51 | (Sensor A) | 9.26 | (Sensor A) | 269% |
| | | of pain; much longer | | 3.51 | (Sensor B) | 13.63 | (Sensor B) | 288% |
| | | walking distance. | | | | | | |

In some embodiments, systems and methods can involve a processor configured to calculate a BPI index (e.g., a BPI ratio) and a VHI ratio for each location being measured, and take into account BPI and/or VHI indices above or below predetermined threshold values. As illustrated above in Tables 2 and 3 for example, in the subset of cases where there was an unambiguously positive outcome, significantly large increases in both VHI and BPI were identified. In some embodiments, a processor can be configured to analyze metrics relating to blood flow characteristics, and predict a qualitative and/or quantitative likelihood of healing/improvement, and/or suggest expedited medical follow-up due to limited improvement or a lower likelihood of improvement. The recommendations can be electronically delivered to an output device such as a display as previously described, and be in text or graphic form.

For the outpatient detection of ischemia, VHI can advantageously be used as a diagnostic tool to distinguish clinically ischemic feet from healthy feet. A study of healthy patients vs. patients with clinically diagnosed PAD or CLI, generated the following AUC graphs, illustrated in FIGS. 14E and 14F.

As shown in the left graph of FIG. 14E above, VHI outperforms ABI over all patients. As shown in the right graph of FIG. 14F, in patients with ABI>1.1, VHI maintains its sensitivity, unlike ABI.

The results illustrate that VHI can be far superior to ABI in detecting foot ischemia, especially in cases where the ABI reading is >1.1. In such cases, due to the possibility of highly calcified incompressible ankle vessels, ABI cannot distinguish between healthy tissue and severely ischemic tissue, while VHI maintains its accuracy as it is not affected by calcification.

In one study, the baseline VHI for 20 patients was analyzed as shown in FIG. 14G and as follows.

Across 40 index foot baseline measurements on these 20 patients, 85% was ≤15 VHI while 92.5% was ≤20 VHI. The median VHI was 9.3.

As noted above, VHI can be used to discriminate between healthy feet versus clinically-ischemic feet that require intervention.

For the same patients, the median of the average BPI value over the same 5-minute period (taking a visual estimate of the average BPI off the 5-minute chart) is shown in FIG. 14H.

Across 40 index foot baseline measurements on these 20 patients, 77.5% was ≤30 BPI, and 92.5% was ≤40 BPI. The median BPI was 19.0.

The consistency of the BPI median analysis relative to the VHI median analysis can be seen; the VHI numbers approximate half their BPI equivalent.

As such, a processor configured to determine a VHI threshold level, such as less than about 25, 20, 15, 10, or even less, and/or BPI threshold level of 45, 40, 35, 30, 25, or even less can be used at the frontline to direct patients towards more diagnostic tests with a view towards more timely intervention for limb salvage.

Systems and methods can also be configured to angiosome specificity—for example, can be configured to track perfusion changes in different topographical areas of the foot.

In some embodiments, intraoperative monitoring utilizing perfusion monitoring using systems and methods as disclosed herein can provide a more aggressive procedural strategy to address difficult lesions/CTOs, given the flat or reduced BPI at procedure end. This could result in an avoidance of an emergency re-admission or repeat amputations. Furthermore, aggressive reperfusion strategies guided by real-time perfusion feedback may help to reduce the cost (both in terms of expense and patient outcome) associated with repeat revascularization.

In some embodiments, post-op perfusion monitoring can be used to alert the clinician as to the need for further intervention or anti-coagulants/thrombolytics to head off soft thrombus issues before they cause a downward spiral of events. Early follow up utilizing systems and methods configured to determine indices such as those disclosed herein could determine either a fall or no change in perfusion, and this can be taken into account by the clinical team when deciding on further clinical treatment, including whether to repeat the intervention.

In addition to the above-described real-time monitoring of blood perfusion in the operating room, derivative indices based on the raw blood perfusion data generated via DCS or DSCA can also serve as tools in an inpatient or outpatient setting, for example, to direct appropriate wound or ulcer therapy based on the patient's level of tissue perfusion, or to screen for critical thresholds of peripheral arterial disease, by measuring blood perfusion in the extremities (e.g. the foot). Such derivative indices include the Foot Thumb Index (“FTI”), the Low Frequency Oscillation Index (“LFI”) and its two parameters of “LFIA” and “LFI_{M}”, as well as the Support Vector Machines Index (“SVM”) and the Flow Transform Level (“FTL”). These derivative indices are described below and will jointly be referred to as “the Derivative Indices.” In some embodiments, the function of time references in one or more of the derivative indices can be, for example, between about 15 seconds and about 15 minutes, between about 30 seconds and about 5 minutes, between about 30 second and about 2 minutes, or about 30 seconds, 45 seconds, 1 minute, 1.5 minutes, 2 minutes, 2.5 minutes, 3 minutes, 4 minutes, 5 minutes, 6 minutes, 7 minutes, 8 minutes, 9 minutes, 10 minutes, or ranges involving any two of the foregoing values.

Statistical Analysis of a BFI signal:

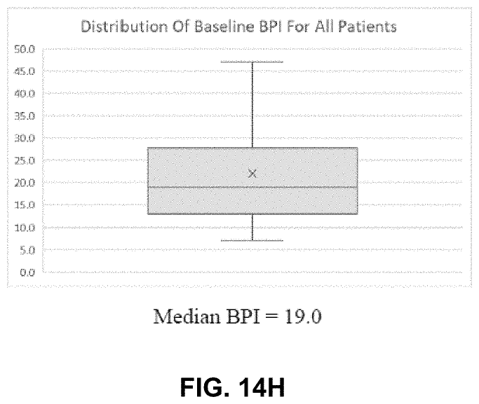

In some embodiments, the statistical parameters of the BFI (BPI) signal can also be used as a discriminator. The Flow Transform Level “FTL” is the standard deviation of the BFI signal calculated at 2 Hz. FIG. 12 shows how this is derived from and relates to the time series BFI, e.g., derivation of FM from time series DSCA blood flow index (BFI) data, where intensity is measured at a frame rate of 60 Hz. Other frame rates, such as 30 Hz for example, can also be utilized depending on the time duration selected.

The standard deviation of 5 minutes of Medial Plantar BFI data sampled at 1 Hz and 2 Hz was calculated, and the resulting ROC curves are shown in FIGS. 13A and 13B. FIG. 13A illustrates the ROC of Standard Deviation of BFI 1 Hz; FIG. 13B illustrates the ROC of Standard Deviation of BFI @ 2 Hz. As noted elsewhere herein, the amount of time data sampled can be selected depending on the desired clinical result, such as about 30 seconds, 45 seconds, 1 minute, 75 seconds, 90 seconds, 105 seconds, 2 minutes, 3 minutes, 4 minutes, 5 minutes, or another time interval. Other frequencies other than 1 Hz or 2 Hz can be utilized as well, such as a frequency of between about 0.5 Hz an about 10 Hz, or between about 1 Hz and about 10 Hz.

If the standard deviation of the BFI at 2 Hz is focused on, and the data set shortened and analyzed, a slow degradation of the AUC down to 2 minutes can be observed, and a precipitous drop at 1 minute. This result is shown in Table 3A.

TABLE 3A
Dependence of FTL AUC on sample time/data set size.

| Sample time | AUC for FTL |
|---|---|
| 5 min | 0.9645 |
| 4 min | 0.9633 |
| 3 min | 0.9554 |
| 2 min | 0.9241 |
| 1 min | 0.7428 |

The Standard Deviation of BFI from calcaneal and arm also shows significant difference between healthy and ischemic patients, but not strongly as with the medial plantar. The p-values of three positions are compared in FIGS. 14A-14C, which are box plots of FTLs in the medial plantar, calcaneal, and arm regions, respectively.

### Assessment of Results

An AUC of the ROC curves of around 0.75 or higher showing a decent discriminating power, and an AUC exceeding 0.90 is considered excellent in some embodiments. By way of comparison, Figoni et al (J. Rehab Res Dev 2006: 43 (7) 891-904) report that tcPO2 has an AUC of 0.82 in discriminating between healthy subjects, and ischemic patients (identified as prospective candidates where unilateral transtibial amputation was imminent or scheduled because of lower-limb ischemia). The ischemic group of patients in the Figoni study however suffered from an extreme degree of ischemia in that the decision for an amputation at a level much above the site of TcPO2 measurement had already been made. In some embodiments, patients analyzed are typical patients in an out-patient setting, with none requiring amputations at the time of testing. FIG. 14D illustrates a graph showing FTL values obtained in one study for healthy and ischemic patients on the Y axis and the patient numerical identifier on the X axis.

Despite this difference in the degree of ischemia between subjects with respect to the Figoni study, one-dimensional AUC using LFI_{M} can be similar to the Figoni study suggesting a much greater ability for LFI to distinguish subtle differences in the degree of ischemia compared to TcPO2. When utilizing multiple parameters in our SVM, an AUC of 0.969 or better can be achieved, far exceeding the performance reported for tcPO2.

Using FTL (Standard Deviation of BFI @ 2 Hz) an AUC of 0.9645 with a single parameter can be achieved from a single sensor located at the medial plantar. This greatly simplifies the measurement in some cases and can increase the utility and ease of implementation of technique for clinical diagnostic and/or screening applications.

In some embodiments, an FTL value of less than about 10, 9.75, 9.5, 9.25, 9, 8.75, 8.5, 8.25, 8, 7.75, 7,5, 7.25, 7, 6.75, 6.5, 6.25, 6, 5.75, 5.5, 5.25, 5, 4.75, 4.5, 4.25, 4, 3.75, 3.5, 3.25, 3, 2.75, 2.5, 2.25, 2, or less can serve as a pre-determined discriminatory cut-off value between a first population and a second population and indicate a risk factor for a characteristic or a disease characteristic, e.g., ischemia, such as severe ischemia, and notify the clinician by prompting an audible, visual, or other signal, such as visually on the display, for example.

Some embodiments may also include memory to store measured or computed data (such as but not limited to BFI/BPI, a BPI ratio, raw DOF signals), and the capacity to transmit/receive measured or computed data to/from at least one website/database. The at least one website/database can provide patients and clinicians access to the measured or computed data, process/analyze the data and provide notifications to clinicians and/or patients. These notifications may include, but are not limited to, alerts when patient should seek medical attention, updates to clinicians that new patient data is available for review, etc. The data can be stored in a manner and compliant with standards applicable to electronic health records of hospitals and diabetic/podiatry/geriatric/community care centers. Such a system can enable clinicians, care givers, and family members to remotely monitor patients, and can be especially relevant in resource limited regions where access and travel to clinical care centers are limited and/or difficult. By remotely assessing patient's health, it will be possible to improve clinical care by ensuring that only essential travel is undertaken.

In some embodiments, systems and components as described herein can take the form of a computing system that is in communication with one or more computing systems and/or one or more data sources via one or more networks. The computing system may be used to implement one or more of the systems and methods described herein. While various embodiments illustrating computing systems and components are described herein, it is recognized that the functionality provided for in the components and modules (which may also be referred to herein as engines) of computing system may be combined into fewer components and modules or further separated into additional components and modules. For example, a communications engine may include a first module in communication with a diagnostic imaging modality and a second module in communication with a destination modality. Modules can include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. Any modules can be executed by one or more CPUs.

A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules described herein can be implemented as software modules, but may be also represented in hardware or firmware. Generally, the modules described herein refer to logical modules that may be combined with other modules or divided into sub-modules despite their physical organization or storage. In addition, all the methods described herein may be executed as instructions on a CPU, and may result in the manipulation or transformation of data.

In some embodiments, hardware components of the system includes a CPU, which may include one, two, or more conventional microprocessors. The system further includes a memory, such as random access memory (“RAM”) for temporary storage of information and a read only memory (“ROM”) for permanent storage of information, and a mass storage device, such as a hard drive, flash drive, diskette, or optical media storage device. Typically, the modules of the system are connected using a standard based bus system. In different embodiments, the standard based bus system could be Peripheral Component Interconnect (“PCP”), Microchannel, Small Computer System Interface (“SCSI”), Industrial Standard Architecture (“ISA”) and Extended ISA (“EISA”) architectures, for example.

In accordance with some embodiments, systems may be operatively coupled to a destination modality, such as, for example, an electronic medical record (“EMR”). EMRs may be any software or hardware-software system configured to store and provide access to electronic medical data. In accordance with various embodiments, EMRs may be at least one of an electronic medical record, an electronic health record, and the like. In some embodiments, systems and components thereof can be operatively coupled to a destination modality that can be an email or other messaging modality; SAMBA, Windows, or other file sharing modality; FTP or SFTP server modality; a VPN; a printer; and the like.

In accordance with some embodiments a system may comprise one, two, or more software modules, a logic engine, numerous databases and computer networks configured to provide a user with access to various modalities as described herein and/or an EMR. Systems may be configured such that patient data, or no patient data is recorded by the system. While the system may contemplate upgrades or reconfigurations of existing processing systems, changes to existing databases and business information system tools are not necessarily required. Systems may be implemented or integrated into existing healthcare information management systems, such as EMRs, without changes to the EMR system, and may interface with other modalities without changes to the communication system of the modality.

In accordance with some embodiments, systems may be software or hardware-software systems. For example, systems can include a communication engine configured to receive and transmit medical information operatively coupled to an information converter configured to render diagnostic medical information in a suitable format for storage in a patient EMR; a work list engine configured to create a user selectable task list from orders captured at an EMR and selectable by a user at a medical diagnostic modality; and an event log configured with a user selectable record of transactions and/or errors in data transmission and/or data conversion performed by the system.

In accordance with some embodiments, communication engine may be any software or hardware software-system configured to receive and/or transmit data. Communication engine may be configured to transmit and receive data over a variety of network interfaces including wired and wireless networks or a combination thereof, such as via. Ethernet, 802.11x, Bluetooth, FireWire, GSM, CDMA, LTE, and the like. Communication engine may also be configured to transmit and/or receive data with file transfer protocols such as TCP/IP, as well as various encryption protocols, such as, for example, WEP, WPA, WPA2, and/or the like.

Furthermore, in some embodiments, a communication engine may be configured as an active or passive module. When communication engine is passive, it may be configured to be discoverable by various elements of a larger healthcare management system. In this way, communication engine may be configured to receive a command or request from a medical diagnostic modality for a user selected patient, such that the communication engine may transmit the request to an EMR, receive the patient data for a specific patient from the EMR, and transfer the patient data from the EMR to the medical diagnostic modality. As such, communication engine is only configured to receive and transmit data. In some embodiments, communication engine is not configured to collect, capture, or mine data from, either, an EMR or a medical diagnostic modality.

### Clinical Applications

Embodiments of Derivative Indices of DSCA provide a direct assessment of microvascular vasomotion in the patient. Endothelial dysfunction caused by diabetes (Kolluru et al in Intl J of Vascular Med 2012) undermines normal vasomotion, leading to delayed vascular re-modeling and wound healing. The Derivative Indices therefore can in some embodiments provide means to better assess the healing capacity of patients (both diabetic and non-diabetic) and hence direct the optimal use of wound care therapy. Additional use for the Derivative Indices could be for screening patients for peripheral vascular disease, determining the efficacy of a revascularization procedure, such as a bypass, stent, graft, angioplasty, or other procedure, either intraoperatively or postoperatively; predicting response to advanced wound therapies such as HBOT, and determining the optimal sites for limb amputation, for example. Other applications of this technique include, for example, the assessment of plastic surgery grafts or flaps for tissue viability. In some embodiments, DOE sensors can be used to assess blood flow in the foot, ankle, calf, thigh, hand, arm, neck, or other anatomical locations. In some embodiments, the DOF sensors can be positioned within the body, for example within natural orifices, such as the esophagus, stomach, small intestine, colon, or uterus for example to assess blood flow in various such embodiments, DOE sensors can be disposed in accordance with angiosome theory.

### Ischemic Foot Screening

One, two, or more of the Derivative Indices may be used as a tool to screen for ischemic feet, particularly for diabetic patients where the presence of neuropathy as part of the diabetic disease progression means that claudication is often not a reliable manifestation of the severity of underlying peripheral arterial disease, e.g., the patient feels no pain due to diabetic neuropathy, rather than because there is no atherosclerotic disease.

As a screening tool should ideally be small, compact, inexpensive, and widely deployable and utilized by staff with minimal training, in some embodiments the system for screening ischemic feet may be implemented using a small, battery powered, portable, blood perfusion monitor console comprising a single sensor that is attached to the patient's foot for measurement duration of, for example, 10 seconds to 10 minutes. The recorded time series blood perfusion can then be processed into a power spectrum via an internal processor. Alternatively, the time series data may be telemetered to a distributed computational network for processing. Results of the calculated one or more Derivative Indices can then be reported directly to the physician's office or care giver for further follow-up. Alternatively, caregivers or clinicians may remotely access results via the internet, smart phone, or other telecommunications device. Patients who present with endothelial dysfunction and/or ischemia can then be referred to primary care centers for more directed evaluation and therapy.

Diabetic feet are also at risk of ulceration from a combination of ischemia, high plantar pressures from bio-mechanical change in the foot as well as neuropathy. In clinical practice, the combination of these three factors leads to a diagnosis of a diabetic foot at risk of ulceration (“DFAR”). Annually, 25% of diabetics are thus diagnosed to be at risk of ulceration, and 50% of such diagnosed patients subsequently undergo a major or minor amputation of foot tissue.

Some approaches measure the three diagnostic indicators separately—the ankle-brachial index (“ABI”) can be used to measure ischemia, while a pressure footplate can be used to measure plantar pressure, and a pressure-sensitive monofilament that buckles at a pre-determined pressure but is not felt on application by the patient can be used to diagnose neuropathy. There are multiple disadvantages of these approaches, including (a) ABI measurements are highly variable depending on the procedural protocol that in turn varies from hospital to hospital. The position of the patient is highly material as ankle systolic pressure is affected by posture—1 mmHg higher for each inch the ankle is below the heart; (b) the presence of calcified vessels in diabetic feet can generate falsely high readings of ABI; and (c) the clinic workflow can become congested at the physician's desk as it takes a medically qualified doctor to subjectively interpret on a case-by-case basis three different reports for ischemia., plantar pressure, and neuropathy in order to make a determination of a diabetic foot at risk. It typically takes 30 minutes or more for a physician to run these tests and make a diagnostic determination.

Some embodiments described herein include one, two, or more flow sensors, such as diffuse optical flow (DOF) sensors configured to measure one, two, or more parameters relevant to blood flow, and operably connectable to one, two, or more anatomical regions of interest, such as a foot or hand for example. The sensors are in operative wired or wireless communication with a hardware console unit configured to receive the parameters from the sensors and perform predetermined calculations as described elsewhere herein. Some embodiments described herein comprise a pressure-sensitive footplate into which is embedded at least one diffuse optical flow (DOF) sensor heads which will be in optical communication with an angiosome or other topographic location of the patient's foot so as to take a measurement based on one or more of the Derivative Indices, and, optionally, at least one DOF reference sensor head that can be applied to a suitable location on the patient such as the thumb or the earlobe, to obtain a reference reading for computation of the FTI. The device may generate a quantitative readout per foot of the absolute BFI and/or FTI and/or any other Derivative Index, as well as the plantar pressure, each with objective threshold criteria for indicating whether a foot needs further physician review and therapeutic or pre-emptive intervention. The device represents a simple, objective and intuitive method of diagnosing a diabetic foot at risk of ulcer in a way that removes inter-operator variation and avoids multiple tests. In some embodiments, to generate a report of the relevant data, the patient merely has to stand on the footplate device for a short period of time, for example approximately 30 seconds with an adhesive sensor head affixed to one thumb or other reference point. Such a simple outpatient tool can be easily used by nurses, clinical technicians, physiotherapists etc. in the diabetes or podiatry care community to more efficiently triage diabetic feet at risk and thereby ease the workflow congestion caused by the chronic shortage of physicians in many aging communities worldwide.

### Guiding Wound Management

Current techniques utilized to assess wound healing potential are sub-optimal. TcPO₂ measurements have been shown to be poor predictors of HBOT outcome (Fife et al, Wound Rep Reg 2002; 10: 198-207). Skin perfusion pressures are in fact better predictors of wound healing than TcPO₂ (Lo et al in Wounds 2009), though with a diagnostic accuracy of less than 80% for an SPP cutoff value of <30 mmHg (Castruonuovo et al in JVS 1997).

It is possible that TcPO₂ and SPP will never reach the highest levels of diagnostic accuracy demanded by the clinical community, as both are limited by the fact that measurements are only skin deep. Studies by Rucker et al (Rucker et al in Am J Physiol Heart Circ, 2000) showed that under critical perfusion conditions, it is the vasomotion and flow motion in the skeletal muscle that preserve nutritive function to surrounding tissue like skin, subcutis and periosteum, which are incapable of this protective mechanism. In addition, the impaired endothelial dysfunction as seen in diabetes directly impairs vasomotor function (Kolluru et al in Intl J of Vascular Med 2012) leading to delayed vascular re-modeling and wound healing. It follows therefore that measurement of either just partial pressure of oxygen (TcPO₂) or perfusion pressure in the skin alone (SPP) does not reflect the critical nature of the ischemia in the underlying tissue, and hence provides at best a partial indicator/predictor of wound healing.

In contrast, the Derivative Indices directly measure the vasomotor function in tissue at a depth much greater than skin (up to 2 cm), and thus have the potential to be a superior predictor of wound healing, and a powerful tool to guide the appropriate therapy for wound healing. In some embodiments, blood flow can be measured at a depth of greater than about 2 mm, 4 mm, 6 mm, 8 mm, 10 mm, 12 mm, 14 mm, 16 mm, 18 mm, 20 mm, or more.

Conservative therapy for wounds (e.g. bandages, moist dressings) can suffice to facilitate wound healing if the blood perfusion around the wound tissue is not compromised beyond the minimal threshold for passive healing to occur. In cases where the perfusion is thus compromised, however, the inappropriate use of conservative wound therapy causes a time lag between the first presentment of a wound in a clinical setting to an effective therapy commensurate with the seriousness of the wound condition. The TIME (Tissue viability, Infection control, Moisture, Epithelialization) model of wound care emphasizes the need for early diagnosis of tissue viability or otherwise in a wound, which diagnosis will then drive the therapy pathway towards wound healing. The single most important determinant of tissue viability in a wound is its blood supply. The ability to assess the blood perfusion around. the wound bed allows clinical decisions to be made regarding either (a) continuation of conservative therapy if tissue is viable or, (b) if blood perfusion is too severely compromised for successful conservative therapy, to progress to more advanced wound care products like chemical debriding agents, or advanced wound therapies such as topical negative pressure, hyperbaric oxygen therapy etc. In more serious cases, the patient can be directed to revascularization by peripheral interventional procedures.

### Guiding Amputation Levels

The Derivative Indices may also have a role in predicting the success of amputation healing. Amputation is typically performed on patients with severe limb ischemia who cannot be treated with reconstructive vascular surgery, patients with diabetic foot ulcers or venous ulcerations. Approximately, 85-90% of lower limb amputations in the developed world are caused by peripheral vascular disease and poor wound healing accounts for 70% of the complication cases that arises from amputation. In spite of the use of state of the art technologies to assess amputation level, the healing rate of below-knee amputation ranges between 30 and 92%, with a re-amputation rate of up to 30%. Post-amputation wounds fail to heal if the blood perfusion at the amputation level is inadequate to support wound healing. When this occurs, the surgical wound breaks down, often with superadded infection, and can add to revision amputation where the leg is amputated at a higher level, or to the morbidity of the patient as well delays in patient rehabilitation and prosthetic fitting. The ability to measure blood perfusion using one or more of the Derivative Indices may enable the physician to better predict successful amputation healing at different levels of the leg to be amputated. This will guide the physician via objective criteria as to the appropriate level of amputation to minimize patient pain and suffering while maximizing limb preservation.

In some embodiments, systems and methods can be used for a wide variety of indications, including but not limited to monitoring limb or other target location perfusion and monitoring of overall patient health in inpatient settings (e.g., intensive therapy units, emergency departments, operative suites, and other areas); outpatient settings (e.g., clinics, ambulatory surgical centers, skilled nursing facilities, and home environments); immediate post-operative surveillance for a desired period of time, e.g., overnight following arterial bypass; monitoring how tightly applied compression bandages are applied to patients with lower limb venous ulcers; assessing for skin damage as an early warning for pressure ulcers; monitoring patients' limb perfusion post trauma; assessing skin health prior to non-amputation, orthopedic intervention; monitoring for the development of ischemia during surgery/development of compartment syndrome; monitoring solid organ transplants; implantable myocardial sensors to monitor patient's hearts post-op; implantable brain tissue sensors to monitor for ischemic stroke and/or revascularization procedures; and the like.

The perfusion sensors could be transcutaneous (e.g., without any implantable components), percutaneous, or implanted in some cases depending on the desired clinical result.

In some embodiments, a system can include at least a first sensor, and a second sensor spaced apart from the first sensor at a different anatomic location. The second sensor can be a reference sensor that measures perfusion of tissue different from that measured by the first sensor. Not to be limited by theory, changes in perfusion can be multifactorial, including local effects such as caused by peripheral vascular disease, for example, as well as more systemic changes including vasodilation or vasoconstriction caused by the autonomic nervous system, pharmacologic agents, a change in fluid status, and the like. Such systemic changes can introduce confounding variables not necessarily related to the tissue measured by the first sensor, and obscure whether a change in perfusion is related to, for example, an intervention or rather just a systemic effect.

As such, including one, two, or more reference sensors in a system can advantageously provide data to a controller from sites that are unaffected or substantially unaffected by any local conditions present in the first anatomic location, such as peripheral vascular disease for example, which allows for the system to adjust for non-local effects. In some embodiments, a perfusion index can be adjusted based on input from the reference sensor. For example, a controller can receive inputs from the first sensor and the reference sensor(s), and calculate an adjusted index (e.g., BPI ratio, VHI, or other indices including those disclosed herein) based on a predetermined algorithm, including but not limited to a division calculation (e.g., first sensor value divided by the reference sensor value), subtraction calculation (e.g., reference sensor value minus the first sensor value), and the like.

In some embodiments, the reference sensor(s) can be on a different body part than the first sensor, such as a location in a different vascular distribution than measured by the first sensor. For example, a first sensor can be placed on a lower extremity, such as a foot for example, and the second reference sensor can be placed on an arm, forearm, torso, forehead, or other desired location. As another non-limiting example, a first sensor can be placed on a first lower extremity, and a second sensor placed on a second lower extremity. In some embodiments, the reference sensor can be placed on a contralateral or ipsilateral side of the body as the first sensor. In some embodiments, a system could include more than one reference sensor (e.g., on an arm, and on a torso, for example)

### Screening for Hyperbaric Oxygen Therapy

Hyperbaric oxygen therapy to aid the healing of chronic non-healing wounds is currently directed by the measurement of TcPO₂ in the skin surrounding the wound bed before and after the administration of 100% oxygen. HBOT involves the administering of oxygen at levels 2-2.5 times sea level in a chamber. The administration of HBOT as a therapy over a long period of time is not only expensive and comes with many undesirable side effects such as ear and sinus barotrauma, paranasal sinuses and oxygen toxicity of the central nervous system. (Aviat Space Environ Med. 2000;71(2):119-24.) Moreover, a retrospective study of 1144 patients (Wound Rep Reg 2002; 10:198-207) indicated that 24.4% of chronic wound patients who received HBOT obtained no benefit from it. There is therefore a need to better predict the success of HBOT for any given individual. Since measurements of the Derivative Indices are taken at tissue depths well below skin level, it holds potential for the ability to identify those patients for whom HBOT may well be unsuitable.

### Assessment of Surgical Flaps

A further use of the Derivative Indices in clinical practice lies in surgical procedures, particular in plastic and reconstructive surgery, where pedicled or free tissue flaps are used to cover wound defects. Skin, myocutaneous, fascia-myocutaneous and osseomyocutaneous flaps are used to reconstruct tissue defects that may result from trauma, surgery for tumors, infections or congenital diseases. These flaps depend upon the blood supply from either their own blood vessels or from micro-vascular reconstructions with the blood vessels in the vicinity of the recipient tissue bed for their survival. Both types of flaps (pedicled and free) are crucially dependent on the blood perfusion within them for the flaps to survive. Flap perfusion needs close monitoring especially in the first few hours to days after the reconstruction procedure and early detection of loss of perfusion will help to direct the patient for further surgical procedures as needed to ensure continued flap viability. Monitoring the perfusion of these flaps either via surface sensors or sensors within the flap tissue may guide the physician towards an early intervention that can preserve the viability of the flap. The Derivative Indices can be potentially used to monitor flap blood perfusion continuously in the post-operative period and prevent flap loss due to delayed detection of flap ischemia.

Intravascular and/or Intra-luminal Tissue Probes For Use In Guiding Decisions For Various Therapies

In another embodiment, a DOF sensor for blood flow assessment, e.g., intravascular use comprises at least two fibers configured to emit/receive optical signals at their distal ends, that is delivered via percutaneous and/or transluminal means into an organ or tissue bed that allows for DCS or DSCA measurements of blood perfusion in tissue volumes which are in optical communication with the at least two fibers. Such an intravascular sensor may be configured to have a small cross-section similar to a guidewire of between about 0.01 to about 0.04 inches (or about 250 microns to about 1 mm). The intravascular sensor may be disposed within a flexible sheath that will protect it during delivery, and facilitate insertion of the probe into the target tissue, whereupon the sheath may be partially retracted or the distal tip of the probe partially extended beyond the end of the sheath, so as to put the distal ends of the at least two fibers in optical communication with the tissue whose perfusion is to be measured.

Intravascular and/or intra-luminal tissue probes can enable the real-time measurement of blood perfusion in visceral organs or tissue to guide decisions in various medical therapies, including current treatment protocols for cancer therapy and vascular malformations. These examples are described in greater detail below. In some embodiments, systems and methods as disclosed herein can be utilized for the diagnosis and assessment of the efficacy of various therapeutic interventions for a wide variety of indications, including transient ischemic attacks and acute ischemic strokes (and the efficacy of a neurointerventional revascularization procedure, such as angioplasty or stent placement), ischemic bowel, pulmonary embolism, myocardial infarction, and others. In some embodiments, systems and methods can also measure active bleeding (such as GI bleeding) and confirming the cessation thereof. Other indications are described below.
(a) Measuring tumor vascularity and its impact on photodynamic therapy as well as tumor sensitization measurements before radiofrequency ablation

The following articles refer to the need for assessing tumor blood flow in directing radiotherapy, chemotherapy and photodynamic therapy, and are hereby incorporated by reference in their entireties. (Int, J, Radiation Oncology Biot, Phys 2003 V 55, No 4, pp 1066-1073, “Nitric oxide-mediated increase in tumor blood flow and oxygenation of tumors implanted in muscles stimulated by electric pulses”, B. F. Jordan, Bernard Galles et al; The Oncologist 2008, 13:631-644 “Use of H₂ ¹⁵O-PET and DCE-MRI to Measure tumor blood flow”, Adrianus J de Langen et al,; Radiat Res 2003 October 160 (4) 452-9 “Blood flow dynamics after photodynamic therapy with verteporfin in the RIF-1 tumor” Chen B Pogue, et al) In brief, the potential for success for chemotherapy is higher in well-perfused tumors. Prior knowledge of this can be used to identify those patients likely to respond well to treatment and stream such patients with greater confidence for chemotherapy treatment. Quantitative measurement of tumor blood flow may also help calculate doses of chemotherapeutic agents to be delivered, especially when such chemotherapy is directly delivered into the tumor via intra-luminal or endovascular means, This will help to avoid the unnecessary and painful chemotherapy of patients who are unlikely to benefit from treatment due to the poor vascularity of their tumors.

Perfusion has also been shown to play a key role in the success of hyperthermic treatments like radiotherapy and photodynamic therapy. Oxygen deficiency in tumors has been shown to reduce repose to non-surgical treatment modalities like radiotherapy and chemotherapy. This oxygen deficiency may be caused by decreased tumor perfusion (diffusion-related hypoxia) or changes in red cell flux (acute hypoxia). Increasing tumor perfusion by various methods such as use of vasoactive agents, carbogen breathing and electrical stimulation of skeletal muscle surrounding the tumor to increase tumor blood flow have been shown experimentally to have radiosensitizing effects, Photo-dynamic therapy (PDT) uses the principle of light at specific wavelengths causing damage to tumor vasculature and rendering the tumor ischemic, i.e. starving the tumor of its blood supply. Success of PDT is thus assessed by the extent to which this ischemia is achieved. The ability to measure tumor blood flow either by endovascular or intra-luminal means can thus help direct the use of these methods to enhance tumor response or to assess tumor response to these non-surgical therapies.
(b) Intravascular and/or intra-tissue probes to guide injection of sclerosing and embolic agents during treatment of vascular malformations

Vascular malformations (“VMs”), such as arterio-venous malformations, are a network of abnormal small vessels that are formed spontaneously or occur congenitally or following trauma to create an alternate conduit of blood flow between arteries, veins and capillaries, bypassing the normal blood flow that originates from the artery through the capillary bed of an organ or tissue and thence into the vein. Clinical indications for treatment of a VM include local symptoms of pain, bleeding or ulceration at the site of the VM, and significant cardiac strain (including high output cardiac failure) from the high volumes of blood that flow within these lesions. Superficial VMs may need treatment for cosmetic reasons as well.

The treatment for VMs comprises injection via an endovascular micro-catheter of a sclerosing, agent such as absolute alcohol or sodium tetradecylsulphate, which are toxic to blood vessels and cause sclerosis or scarring that closes up the small vessels within the VM. This may be the sole procedure or as part of a surgical procedure wherein the volume of blood flowing within the VM is reduced prior to surgical excision. Caution is required during this procedure because excessive injection of the sclerosing agent can lead to overflow into normal blood vessels, resulting in significant damage such as skin necrosis, limb loss, acute pulmonary hypertension, or even death. The challenge for the physician is that a balance must be struck between injecting enough sclerosing agent to completely close up the VM, but not so much that the sclerosing agent leaks out and causes serious damage elsewhere. Real-time perfusion monitoring of the VM can signal when blood flow has ceased within the VM or reduced sufficiently to allow surgical resection without significant loss of blood. This may instruct the physician that enough sclerosing agent has been injected and to avoid further injection, thereby reducing the risk of an adverse outcome.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “discriminating between two populations” includes “instructing the discriminating between two populations.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

1-30. (canceled)

31. A computer-implemented real-time method for determining success of a revascularization procedure and/or wound healing of a patient, the method comprising:
measuring blood perfusion characteristics utilizing diffuse speckle contrast analysis (DSCA);
determining a blood perfusion index (BPI) at a first point in time, determining the blood perfusion index (BPI) at a second point in time, determining a blood perfusion index (BPI) ratio from the BPI by dividing the BPI at the second point in time by the BPI at the first point in time;
analyzing the BPI ratio with respect to additional patient characteristics; and
outputting to a display the BPI ratio and indicia of a likely positive or negative patient outcome.

32. The method of claim 31, wherein the additional patient characteristics comprise an absolute value of the BPI.

33. The method of claim 31, comprising outputting to a display indicia of a likely positive patient outcome if the BPI ratio is greater than about 1.

34. The method of claim 31, comprising outputting to a display indicia of a likely positive patient outcome if the BPI ratio is greater than about 2.

35. The method of claim 31, comprising outputting to a display indicia of a likely negative patient outcome if the BPI ratio is less than about 1.

36. The method of claim 31, wherein the first point in time is within 5 minutes of a first angioplasty attempt within the revascularization procedure, and after guidewire placement.

37. The method of claim 31, wherein the second point in time is within 5 minutes of completion of the revascularization procedure.

38. The method of claim 31, comprising outputting to a display indicia of a likely positive patient outcome if the BPI ratio is greater than about 1.1.

39. The method of claim 31, comprising outputting to a display indicia of a likely positive patient outcome if the BPI ratio is greater than about 1.2.

40. The method of claim 31, comprising outputting to a display indicia of a likely positive patient outcome if the BPI ratio is less than about 0.9.

41. The method of claim 31, comprising outputting to a display indicia of a likely positive patient outcome if the BPI ratio is less than about 0.8.

42. The method of claim 31, wherein measuring occurs on the skin surface of the patient.

43. The method of claim 31,
wherein measuring and determining is performed at a location of interest on the patient,
wherein the additional patient characteristics are determined by:
identifying a reference location on the patient at a location perfused by different vasculature than the location of interest on the patient;
measuring blood perfusion characteristics utilizing diffuse speckle contrast analysis (DSCA) at the reference location;
determining a blood perfusion index (BPI) at the first point in time at the reference location,
determining the blood perfusion index (BPI) at the second point in time at the reference location,
determining a reference blood perfusion index (BPI) ratio from the BPI by dividing the BPI at the second point in time by the BPI at the first point in time,
wherein analyzing the BPI ratio further comprises adjusting the BPI ratio at the location of interest based at least in part by the reference BPI.

44. A computer-implemented real-time system for determining success of a revascularization procedure and/or wound healing of a patient, the system comprising:
a laser light source;
a detector; and
a processor configured to electronically perform the following:
receiving measured blood perfusion characteristics from a location of interest utilizing diffuse speckle contrast analysis (DSCA) from the detector;
determining a blood perfusion index (BPI) at a first point in time,
determining the blood perfusion index (BPI) at a second point in time,
determining a blood perfusion index (BPI) ratio from the BPI by dividing the BPI at the second point in time by the BPI at the first point in time;
analyzing the BPI ratio with respect to additional patient characteristics; and
outputting to a display and electronically indicating the BPI ratio and indicia of a likely positive or negative patient outcome.

45. The system of claim 44, wherein the additional patient characteristics comprise an absolute value of the BPI.

46. The system of claim 44, wherein the processor is configured to output to a display indicia of a likely positive patient outcome if the BPI ratio is greater than about 1.

47. The system of claim 44, wherein the processor is configured to output to a display indicia of a likely positive patient outcome if the BPI ratio is greater than about 2.

48. The system of claim 44, wherein the processor is configured to output to a display indicia of a likely negative patient outcome if the BPI ratio is less than about 1.

49. A computer-implemented real-time method for determining necessity of a revascularization procedure, the method comprising:
measuring blood perfusion characteristics utilizing diffuse speckle contrast analysis (DSCA);
determining a blood perfusion index (BPI) over a set period of time;
deriving a vascular health index (VHI) from the BPI data;
analyzing the VHI with respect to additional patient characteristics; and
outputting to a display the VHI and indicia of a likely need for the revascularization procedure.

50. The method of claim 49, wherein the additional patient characteristics comprises an absolute value of the BPI.
